(19) United States
(12) Patent Application Publication
(10) Pub. No.: US 2004/0146615 A1
(43) Pub. Date: Jul. 29, 2004

(54) Processes for evaluating agricultural and/or food materials; applications;and, products

(76) Inventors: John Thomas McDonald, Memphis, TN (US); Brian Benjamin Anderson, Cordova, TN (US); Richard Gene Kaecher, Bartlett, TN (US); Sean Acie Smith, Memphis, TN (US)

Correspondence Address: MERCHANT & GOULD PC, P.O. BOX 2903, MINNEAPOLIS, MN, 55402-0903, US

(21) Appl. No.: 10/474,087

(22) PCT Filed: Apr. 11, 2002
(86) PCT No.: PCT/US02/11511

(30) Foreign Application Priority Data
Apr. 13, 2001 (US) 60283922

(51) Int. Cl. G01N 33/02

(52) U.S. Cl. 426/231

(57) ABSTRACT

Methods of conducting NIR chemical imaging evaluations are provided. The methods are useable to evaluate chemical distribution in agricultural or food applications. Specific examples of such studies are provided. One example involves evaluating a cellulose fiber paper substrate, for distribution therein of a seed based fiber additive. Products from such methods are provided.

### CROSS-REFERENCE TO RELATED APPLICATION

Priority for the current application is claimed from U.S. provisional application Ser. No. 60/283,922 filed Apr. 13, 2001. The complete disclosure of U.S. provisional Ser. No. 60/283,922 is incorporated herein by reference.

### FIELD OF THE INVENTION

The present invention relates to processes for evaluating agricultural materials and products, and food materials and products. In general, the processes involve, among other things, conduct of chemical imaging analyses, for example to facilitate processes and decision making regarding various agricultural and food materials and processes. The invention also concerns: products from such analyses, and, materials as characterized by such analyses.

### BACKGROUND OF THE INVENTION

The agricultural industries and food industries often involve processes concerning complex materials and substrates. It is an ongoing issue for such industries, to be able to develop processes for evaluation of such materials and substrates and the actual effects of modifications on such materials and substrates.

### SUMMARY OF THE INVENTION

According to the present disclosure, techniques are provided for evaluating samples. The applications concern methods or processes for evaluating agricultural and/or food materials. In general, the techniques involve evaluating distribution of a chemical characteristic (component) in a composition. In application, the techniques may involve, for example: (1) evaluating distribution of a chemical in a chemically non-homogenous composition; (2) evaluating distribution of an additive in a composition; or (3) evaluating compositions with respect to modifications made thereto. An example described herein in detail is a process for evaluating distribution of a chemical characteristic (and thus of a component) in an agricultural or food composition. Thus, the evaluation indicates how the component is distributed in the overall composition.

In many applications, processes or methods according to the present disclosure will be conducted in the form of comparatives. For example, the study can be conducted on first and second samples, the first sample being of a composition with a selected additive or component, the second sample being of a comparative. In this context, the term “comparative” is meant to refer to a sample of known content, used for comparison. A typical comparative would be the same composition as the first sample, but prepared without the additive or component.

Techniques described herein can also be used to monitor processes, and to evaluate, quantitatively, the presence of a material in or on a substrate or composition.

In many instances, application and techniques described herein are processes or methods which result in the generation of a chemical morphology NIR-based image contrast plot. In general, such a plot is an image plot derived from spectroscopic imaging data collected in the NIR (near infrared region of light) from a sample, in which the image is presented to display contrast based on differences in chemical moiety distribution within the sample; the contrast being a result of differences in interaction with NIR light by different regions within the sample.

The term “chemical morphology NIR-based image contrast plot” is intended to address such plots no matter how the data is displayed; for example, without regard to whether the data is projected on a screen, printed on a paper, or photographically created. In addition, the term is intended to include such plots no matter how the analytical data sample is managed for display; for example, the display might be: an image of an object at a specific wavelength, an indexed image where the index level is proportional to concentration of a chemical moiety, an image derived from principal component analysis (PCA), or an image derived from some other basic standard spectroscopic technique of data processing, for example, ratio presentation, derivative presentation or normalization. Various approaches (as examples) to generation of chemical morphology NIR-based image contrast plots are presented hereinbelow, and in the examples.

### BRIEF DESCRIPTION OF THE DRAWINGS

FIG. 1 is an SEM (100×) of a cellulose fiber paper substrate.

FIG. 2 is an SEM (100×) of a cellulose fiber paper substrate made similarly to the substrate of FIG. 1, but including therein an agricultural additive.

FIG. 3 is an SEM depiction (800×) of the same sample as shown in FIG. 1.

FIG. 4 is an SEM depiction (800×) of the same sample as that depicted in FIG. 2.

FIG. 5 is a chemical morphology NIR-based image contrast plot, PC3, of a cellulose fiber paper substrate without an agricultural additive.

FIG. 6 is a chemical morphology NIR-based image contrast plot showing a cellulose fiber substrate having added thereto an agricultural additive, as described in Example 1.

FIG. 7 shows a bulk FTIR spectra of: a cellulose fiber paper, without an agricultural additive; and, a cellulose fiber paper with an agricultural additive.

FIG. 8 is a Bright field image (20×) of a cocoa sample from Experiment 2.

FIG. 9 is a chemical morphology NIR-based image contrast plot indicating fat distribution, in the cocoa sample of FIG. 8.

FIG. 10 is a Bright field image (20×) of a second cocoa sample from Experiment 2.

FIG. 11 is a chemical morphology NIR-based image contrast plot showing fat distribution in the cocoa sample of FIG. 10.

FIG. 12 is a transmission Bright field image of a first chicken skin emulsion from Example 3.

FIG. 13 is a Bright field transmission image of a second chicken skin emulsion, from Example 3.

FIG. 14 is a chemical morphology NIR-based image contrast plot of the first chicken skin emulsion of FIG. 12.

FIG. 15 is a chemical morphology NIR-based image contrast plot of the second chicken skin emulsion of FIG. 13.

FIG. 16 is an NIR macroscopic average field of view spectra of five cocoa powders, used in Example 4.

FIG. 17 is an agglomerated reference sample, microscopic NIR sucrose map, from Example 4.

FIG. 18 is an agglomerated pilot sample, microscopic NIR sucrose map, from Example 4.

FIG. 19 is an agglomerated reference sample, macroscopic NIR sucrose map, from Example 4.

FIG. 20 is an agglomerated pilot sample, macroscopic NIR sucrose map, from Example 4.

FIG. 21 is visible grayscale image of raw rib eye steak, from Example 5.

FIG. 22 is a grayscale image of raw rib eye steak taken from the 1214 nm image slice from the hyperspectral image of beef, from Example 5.

FIG. 23 is a visible grayscale image of cooked rib eye steak, from Example 5.

FIG. 24 is a grayscale image of cooked rib eye steak taken from the 1214 nm image slice from the hyperspectral image of beef, from Example 5.

FIG. 25 is an NIR normalized image (1380 nm) of barley grains from sample RE 00-32, Example 6.

FIG. 26 is a digital RGB image of barley kernels from sample RE 00-32, depicted ridge side down, from Example 6.

FIG. 27 is a digital RGB image of barley kernels from sample RE 00-32, ridge side up, Example 6.

FIG. 28 is a digital RGB image of barley kernels from sample RE 00-33, shown ridge down, from Example 6.

FIG. 29 is a digital RGB image of barley kernels from sample RE 00-33, shown ridge side up, from Example 6.

FIG. 30 are barley sample images derived from NIR absorption image cubes, corresponding to chemical morphology NIR-based image contrast plots, from Example 6.

### DETAILED DESCRIPTION

I. Application of Chemical Imaging Techniques in the Agricultural and Food Industries

### A. Chemical Imaging vs. Traditional Surface Contrast Imaging—Generally

Imaging Systems and Domain Size.

Optical imaging techniques rely on the interaction of visible and non-visible radiation with matter to record images of objects. A given imaging system utilizes optical elements, known as lenses, to refract light in a defined manner according to physical laws governing the transmission of light, or energy, through matter. The design and manufacture of lens elements for operation in the visible portion of the electromagnetic spectrum is an engineering field with commercially available products for imaging systems, for example, including consumer photographic cameras, cinematic projection systems, microscopes, and similar systems.

A lens system will form an image in a specific plane at a specified distance from the surface of the lens or optical center of the lens assembly. In some cases, the lens system forms an image that is a 1:1 projection of the object, meaning that there is no magnification of the object. If magnification occurs due to the design of the lens system, then the object is said to be magnified in the image plane. This may result in a decrease in the image size relative to the object, or an increase, depending upon the design goals of the imaging system.

Detector elements are used to record the image formed at the image plane of a given lens or optical system. The classical detector in a visible microscopy system is the human eye, where the optical system projects an image of the object onto the pupil of the eye through the binocular eyepieces, allowing direct visualization of the object under inspection. A goal in microscopy is the magnification of a given object to make features that are too small to be resolved by the unaided eye visible. This is achieved through the use of objective lenses that produce magnifications of 5 times (5×) through 200 times and higher. In addition, imaging systems utilize solid state semiconductor image arrays to record the image via the transduction of the optical energy to electrical energy. Charge Coupled Devices (CCD's) and other array type detectors are used frequently in the recording of images from optical systems. Images from these types of detectors are referred to as digital images due to the digitization of the field of view into individual pixels.

A goal of any imaging system is to transfer the image of the object at a specific magnification to a detector placed at the image plane of the imaging system. The design of the system will be such that the object will be magnified to a greater size, as in the case of a microscope system, or to a much smaller size, as in the case of a 35 mm wide angle photographic camera system.

Herein, an image is considered microscopic, if the field evaluated is less than 3 mm by 3 mm (9 sq. mm) in size. A typical microscope with a standard CCD detector attached in a position designed to accept such a detector will allow for a range of fields of view to be imaged. The field of view for an imaging system is the region of an object that is imaged onto the detector. In the case of a 5× objective used in a visible microscope having a standard ⅔ inch format CCD array, the field of view is approximately 2 mm by 3 mm. Most microscopy systems will use a 5× objective as the low magnification objective. A microscope having a 100× objective will allow a 100 by 150 micrometer field to be viewed. Current imaging technologies, such as atomic force microscopy, allow for features as small as 1 nanometer (1 billionth of a meter) and below to be imaged. Therefore, the microscopic domain by this definition reaches from the upper limit of 3 mm by 3 mm to the lower limit defined by the state of the art in imaging technology.

By utilizing a different optical design, imaging systems can also form images of objects at reduced magnification ratios, i.e., they may form an image that is significantly smaller than the object imaged. For the purposes of this disclosure, the macroscopic domain is defined as ranging from 3 mm by 3 mm (9 sq. mm) to approximately 50 cm by 50 cm (an aspect ratio of 1 is assumed here), a size determined by fixed focal length (non-zoom) lenses for use in the near infrared region of the electromagnetic spectrum.

Much of this disclosure specifically discusses microscopic techniques. However, the techniques for data acquisition, data treatment, and interpretation are identical for macroscopic imaging (as defined) with respect to the fundamental theories governing the generalized imaging experiment. Indeed, examples of imaging both in the microscopic and macroscopic domains are provided, and the techniques presented herein can be applied to both.

1. Traditional Surface Contrast Imaging

Traditional microscopic imaging techniques generally involve a microscopic evaluation of the surface properties of the sample, to obtain simple surface morphology information. Such techniques include, for example, electron beam methods such as scanning electron microscopy (SEM), transmission electron microscopy (TEM) and traditional light methods such as Bright field microscopy. These techniques produce image contrasts generated by the surface properties (i.e., physical geography and topology) of an evaluated substrate.

In many instances, when a substrate has been chemically modified or enhanced, the enhancements do not generate surface morphological (or topological) changes readily observable using such traditional imaging methods, i.e., by surface (or physical) morphology contrast methods. In addition, in some instances, even though differences may be observable, it is difficult to correlate the observed differences to any particular chemical modifications or effects that were induced.

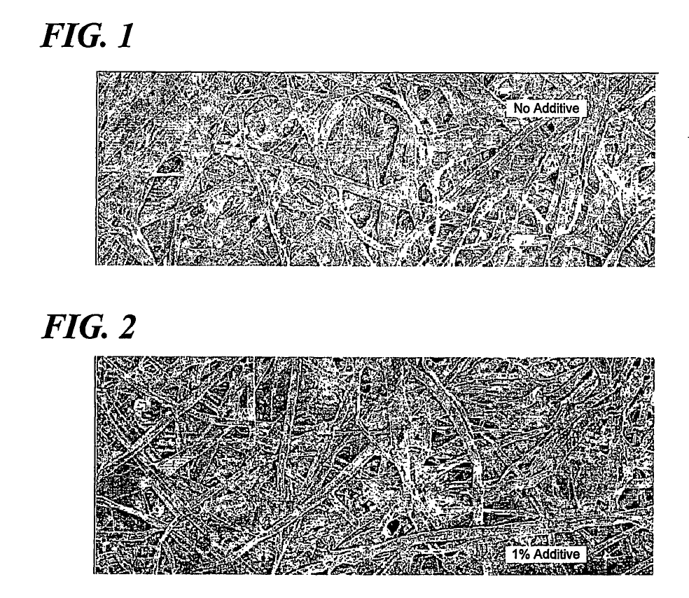

As an example, consider the evaluation indicated in FIGS. 1 and 2 and discussed below at Example 1. Each of FIGS. 1 and 2 is a scanning electron micrograph (SEM) of paper (100×). In general, the process used to prepare each sample was a wet laid cellulose fiber (pulp) process. Indeed, as one would expect, each SEM at 100× shows a cellulose fiber construction.

The sample depicted in FIG. 1 is of a paper sample which has not been modified (by an additive) in the manner described in Example 1 below. The image of FIG. 2 is of a paper sample which was prepared with a desired modification (modified by addition of a seed based fiber material) for evaluation. There are no readily apparent morphological differences viewable from the scanning electron micrographs of FIGS. 1 and 2, even though the two papers would show readily observable differences in physical properties, for example burst strength.

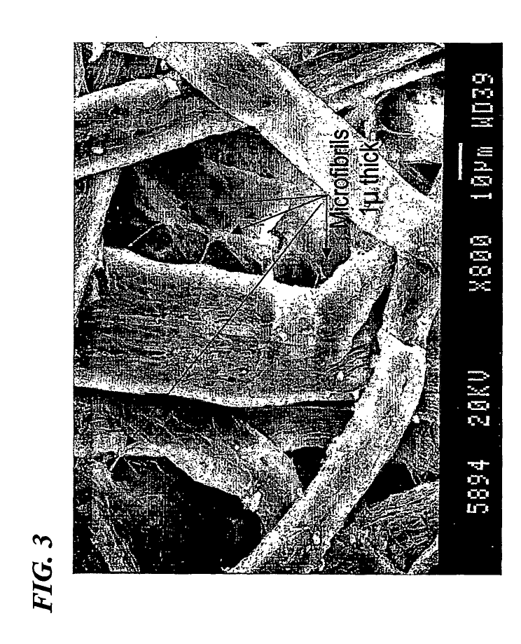

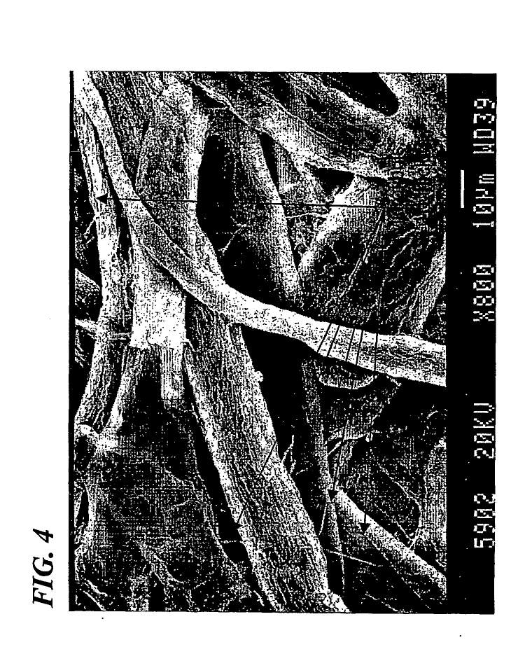

In many instances, efforts to evaluate differences between such types of samples have led to efforts to merely obtain greater and greater magnification, to see if morphological differences (which might explain observed results such as increased burst strength) can be detected. With respect to this, attention is directed to FIGS. 3 and 4. FIG. 3 is an SEM of the same type of material as shown in FIG. 1, but shown at 800× magnification; and, FIG. 4 is of the same type of material as shown in FIG. 2, but at 800× magnification.

When such increases in magnification were made, for the particular samples depicted, several observations were apparent:

(a) First, the 800× evaluation was of only a very tiny portion (about 90 microns by 130 microns) of the substrate (paper sample) and it is sometimes difficult to know to what extent the evaluation is indicative of the substrate as a whole; and,

(b) Although certain morphological differences were discernable, they were relatively fine structural differences and it is difficult to fully evaluate them to understand their extent and cause, as such relates to the fiber additive.

(c) The technique is severely limited due to the inability to discern certain types of differences that do not create strong, visible, structural contrasts.

2. Chemical Imaging

With chemical imaging, instead of merely evaluating surface morphological differences, evaluations are made of localized, discernable, chemical differences. In certain instances, these can be made more readily observable, and sometimes can be made observable on a larger scale, than are surface morphological features. In some instances, the localized chemical differences can be correlated to observable morphological differences, even relatively fine ones.

Problems one might associate with the concept of chemical imaging of samples such as those of FIGS. 1-4, relate to the complex nature of the samples and the chemical nature of the additive. For example, the paper samples are complex in at least the following manners:

(a) They are not of homogenous physical construction, i.e., they comprise randomly laid fibers and thus have open spaces or gaps between the fibers observable on a microscopic level.

(b) The chemical makeup of the fibers, cellulose, is of that a complex polymer.

(c) The additive of concern, seed based fiber, is chemically complex and is also chemically quite similar to the cellulose fibers of the paper.

In spite of these issues, the techniques described herein have provided for an approach to chemical imaging that could be utilized to provide a good understanding of the differences in chemical morphology between the two samples.

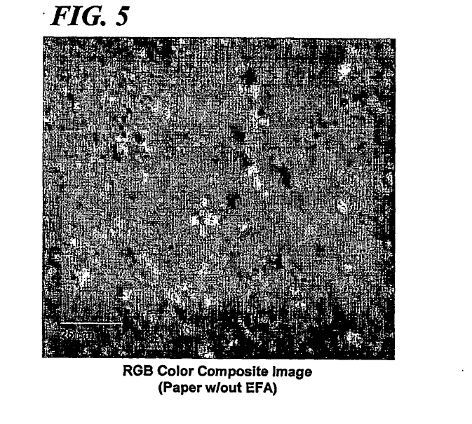

An example of such chemical imaging will be understood by consideration of FIGS. 5 and 6. (It is noted that when the original experiment to create FIGS. 5 and 6 was performed, the plots (RGB color composite images) were made in color, to enhance contrast. The non-color versions presented herein, adequately show the contrast for explanatory purposes.)

FIG. 5 is a plot of a chemical imaging evaluation of a paper sample similar to the one for which images are depicted in FIGS. 1 and 3 above. The plot of FIG. 5 is of a region approximately 330 microns by 330 microns. Herein, the term “330 micron by 330 micron” may alternatively be characterized as a “330 micron square region” or a 108,900 square micron region. The types of plots presented in FIGS. 5 and 6 are a sample of what is generally referred to herein as “chemical morphology NIR-based image contrast plots”. Techniques for producing such plots are described herein below.

From a review of FIG. 5, it is apparent that with respect to the chemical features being analyzed and plotted, the sample is basically homogeneous. Alternately stated, FIG. 5 represents the result of imaging chemical features (or chemical moieties) of the sample and it is noted that with respect to the particular probe or chemical feature that was being evaluated, the sample was homogenous. That is, either the chemical feature(s) (i.e., moieties) being evaluated was not present in the sample, or it was present but distributed such that there were no significant localized differences.

FIG. 6, on the other hand, is a depiction using a chemical imaging approach that was the same as was used to generate FIG. 5, except of a sample made similarly to the ones depicted in the SEMs of FIGS. 2 and 4. Imaged chemical heterogeneity is readily apparent in FIG. 6. Indeed, the image of FIG. 6 strongly shows chemical differences with respect to the chemical moiety imaged, with distribution along the cellulose fiber morphology even though surface differences were not as readily discernable in the SEM. It is again noted that the area of sample imaged in FIG. 6 is about 330 microns by 330 microns.

Already possessing a general understanding of how the samples were prepared and imaged, an investigator provided with FIGS. 5 and 6 can readily conclude that the seed based fiber additive used in preparation of the paper sample of FIG. 6 became greatly distributed along the cellulose fibers, as opposed to between them. From this and the chemical nature of the additive, the investigator can draw conclusions regarding such issues as: how the additive operates; and, what types of alternate additives (or alternative amounts of additive or method of addition) can provide beneficial effects.

Thus, by applying the imaging techniques described herein an investigator could examine how the chemical modifications to the sample actually affected the sample, including how those modifications were localized. These significant observations can then be used in a variety of ways, for example:

1. The distribution of the observable chemical modification (moiety), in concert with an understanding of material morphology, can be correlated to observable chemical or physical properties resulting from the modification.

2. The observable chemical modification, from the imaging, can help an investigator to postulate the manner in which the chemical modification affected the chemical and physical properties of the sample.

3. The information can be used to identify alternate but similar chemical modifications that would be expected to provide similar modifications in physical or chemical properties.

4. The potential impact of the modifications on other material properties can be considered and investigated.

5. The modifications evaluated by chemical imaging can be correlated to otherwise hard to distinguish, fine, differentiations in visibly observable surface morphology.

### B. Selected Issues Relating to Uses for Imaging of Chemical Distribution in the Agricultural and Food Industries.

In section I.A above, an example from the papermaking industry was briefly characterized to provide a background understanding of the differences between chemical imaging and surface morphology imaging. The example of papermaking was selected from what will be generally referred to herein as the use of an agricultural material or additive. As will be understood from reviewing Example 1 below, the actual chemical modification was the result of adding an agricultural material (a modified seed fiber product) as an additive to the wet end of a papermaking process.

In general, in the agricultural and food industries, it can be desirable to evaluate materials, and to monitor and evaluate processes, for example, in the following manners:

1. By evaluating distribution and/or chemical character of a component in a sample;

2. By comparative studies;

3. By monitoring studies; and/or

4. By standard addition/modification studies.

(The various categories in the above list are not meant to be mutually exclusive.)

Types of Studies

1. Evaluating Distribution and/or Chemical Character of a Component in a Sample

In this type of study, a component of a sample is evaluated with respect to its distribution in the sample. For example, an additive in a mixture may be evaluated for its distribution in the mixture. This type of distribution was discussed above in connection with FIGS. 1-6. Another type of distribution study would involve, for example, evaluating distribution of crystal formation in a material such as ice cream; or, evaluation of fat distribution in a material such as chocolate. Other examples would be: mapping uptake of, or treatment by addition of, a treatment agent (such as herbicide(s), pesticide(s) or fertilizer(s)) in plants; and, lignin mapping in fibrous materials.

When reference is made to evaluating the “chemical character” of a component in a sample, it is meant that by utilizing the chemical imaging technique, information about the chemical nature of the distributed material or moiety (or its effect) can be obtained, since the chemical imaging is, in part, based upon locating and imaging a type of chemical characteristic or moiety.

2. Comparative Studies.

(a) Additives.

These types of studies generally relate to evaluating the distribution of an additive in, or on, a substrate or composition; for example, an additive to a food or agricultural product (or material), or the addition of a food or agricultural product or material (additive) to a substrate or composition. An example of an additive study was discussed above in connection with FIGS. 1-6. In that instance, an agricultural product (modified seed based fiber) was added to a substrate or composition (the wet-end slurry of a paper making operation, and ultimately the resulting paper composition). The study involved understanding the agricultural additive distribution in the resulting composition and examining the effects of the addition on the properties of the resulting composition. A mapping of a treatment agent, or the effect of a treatment agent, as characterized in the previous section, would be another example of an additive study.

(b) Other Chemical/Mechanical Treatments of a Substrate or Composition.

In general, these types of studies involve evaluating the effect of modifying or perturbing an agricultural or food substrate or composition via a chemical and/or mechanical process. (Herein the term “chemical process” is intended to include within its scope biochemical processes unless specifically characterized as non-biochemical.) Examples of such modifications would include, for example:

(i) Extractions or other chemical treatments;

(ii) The result of growth;

(iii) The result of deterioration; and/or

(iv) The result of mechanical treatment.

An example of an extraction study, identified at (i), would concern, for example, extracting an identified sample or substrate (for example with water, an organic solvent or a solution such as an acid or base solution, etc.), and then evaluating the substrate or composition treated to determine localized effects or structural effects from the extraction. The following list provides some examples of such studies:

1. Evaluating residual oil seed material (for example, corn) after extraction of oil;

2. Evaluating residual corn fiber, after extraction of lignin;

3. Evaluating residual orange (or other fruit) peel after extraction of flavor components therefrom;

4. Evaluating residual plant material, after extraction of neutraceutical components therefrom; and,

5. Evaluating the effects of modifications in temperature and pH on treatments of a substrate, with respect to distribution of chemical effects therein.

An example of chemical treatment could involve, for example evaluating the treatment of a plant with herbicide, pesticide or fertilizer.

The reference in (ii) to the result of growth, is meant to refer to evaluating a living, growing, or developing substrate or material with respect to chemical morphology, to evaluate the growth or growth pattern. Examples would be evaluating malting processes and microorganism growth in such processes as fermentation processes.

Examples of deterioration studies, characterized generally at (iii), include, for example, evaluating the breakdown of a substrate with respect to chemical morphology, in order to evaluate degradative patterns. Such studies could involve, for example, evaluating the staling or browning/spoiling of bread and other foods; and, evaluating cellular degradation of plant material.

An evaluation of the type indicated at (iv) could involve, for example, evaluating particle shape of various ground agricultural products such as flour, whether from a wet or dry milling process, to examine protein and/or starch distribution in the particles.

3. Monitoring Studies

In monitoring studies, typically an evaluation of a material is made to evaluate quality, progress of process, etc., typically by comparison to either previously generated standards or to a previous evaluation in time. Examples of such studies would include:

(a) Monitoring change in the amount (or relative build-up or loss) of a component in a material; or

(b) Monitoring an identified chemical morphology or distribution characteristic in order to determine whether a resulting product or composition is acceptable or unacceptable, in accord with established standards.

An example of a monitoring study would be evaluating plant samples for different take up (or effects) with respect to herbicide, pesticide or fertilizer addition. Other examples would be monitoring take-up of water or oil by a substrate; or conducting evaluations of fat distribution and quantity, for quality control.

4. Standard Addition/Modification Studies

With this type of study, typically standards are added to samples, or standard modifications are made to samples, and these are compared and evaluated. For example, the addition of a standard may be utilized to evaluate, by comparison, the addition of an unknown. That is, the chemical effects from the addition of an unknown, or partially known, additive can be understood and evaluated by comparing them to the effects of a standard. Similar approaches can be used with known modifications to evaluate modifications which are not fully known or fully mapped.

Examples of such studies, for example, would be:

1. Chemically characterizing a paper material, and then adding selected known components to identify unknowns in the paper material; and,

2. Evaluating oil in a grain in part by using known oils as standard additives.

Selected Issues To Be Addressed In a Well Designed Chemical Imaging Study

Many issues needed to be addressed as part of developing a chemical imaging approach applicable for the types of food and agricultural investigations characterized above. For example, the substrates or compositions involved are generally complex and non-homogenous. By “complex” in this context it is meant that the substrates or compositions may comprise a variety of different chemical moieties and characteristics, and are typically comprised of a mixture of materials, typically complex polymers. By “non-homogenous” in this context, it is meant that the substrates are often neither chemically nor physically homogenous. That is, the substrates often vary, on a microscopic scale, with respect to specific chemical characteristics; and, the substrates often vary, on a microscopic scale, with respect to shape, thickness, or other morphological features. Herein, the term “microscopic scale” unless otherwise qualified, is meant to refer to differences observable in or across a region, for example, of about 3 mm by 3 mm (i.e., 3 mm square or 9 sq mm in area) or smaller.

Another issue with respect to evaluating typical agricultural and food materials, for chemical imaging, relates to identification of analytical approaches that provide for an acceptable level of resolution on an appropriate scale to render the evaluations feasible. For example, in some instances the total amount of material for which distribution or image is desired, is a relatively small percent of the total sample composition, by weight. If the technique being used is not relatively sensitive, chemical distribution mapping won't be feasible. In general, for many studies, it will be desired that the sensitivity and resolution capabilities be sufficient so that a component comprising no more than 10%, typically no more than 5%, often on the order of 2% or less (for example 0.1-1.5%) of the total composition mass, can be evaluated and characterized with respect to its distribution. With respect to this, it is noted that the actual chemical characteristic imaged (for example a carbonyl moiety) may itself represent only a very small percent, by weight, of the component for which distribution is mapped. (By the above, it is not meant that the technique can only be applied when the component comprises no more than 10% of the total composition mass; rather it is meant that preferably the technique is one which can handle such levels.)

Another issue of concern with respect to evaluating a specific chemical distribution in an agricultural or food application relates to the fact that in many instances variations in chemistry between the component to be imaged or mapped (and other materials in the sample) are not abrupt. For example, if one considers the paper additive example described in Example 1 below, and characterized above with respect to FIGS. 1-6, the actual chemical (compositional) differences between the substrate (cellulose fiber from pulp) and the additive (a modified seed based fiber) are not large. In general, each substrate comprises wood cellulose fibers (cellulose being a complex carbohydrate polymer of beta-glucosidic residues) with the fibers being about 80-100 microns in diameter. The seed based fiber additive being evaluated for its distribution in the sample comprises only 0.1-1.5% by wt. of total composition; the seed based fiber additive being very small fibers (typically less than 0.05× the large wood—i.e., pulp—cellulose fibers) also primarily of cellulose (and some hemicellulose) materials. While the materials (additive and substrate) possess chemical differences, the similarities are significant and it might have been expected to be difficult to discern identifiable chemical differences with the type of resolution required to achieve an effective mapping or imaging study.

### C. Near infrared STIR) evaluations as an approach to conducting chemical imaging studies concerning agricultural and/or food products.

As is demonstrated by the examples herein, near infrared (NIR) studies provide a viable approach to chemical imaging (or location mapping) for many applications concerning agriculture and/or food subjects. In general, a near infrared study concerns evaluating the absorption or transmission characteristics of a sample, when subjected to infrared radiation, i.e., electromagnetic radiation of wavelengths within the region of 750 nm to 2500 nm (i.e., wave numbers of about 13,333 to 4000 cm⁻¹). In this context, the term “absorption or transmission” is meant to refer to the fact that such a study can be characterized in terms of either electromagnetic radiation absorption or radiation transmission. Alternately stated, typically the sample is exposed to infrared radiation of a selected wavelength, and the amount of electromagnetic radiation transmitted through the sample is measured. From this, a measurement can be made comparing the amount directed to the sample with the amount that passed through the sample. The difference would be the amount of absorption, typically stated as a % of radiation to which the sample was subjected. On the other hand, a ratio of the amount transmitted through the sample, to the amount transmitted to the sample (×100), would indicate % transmission.

In general the concentration of a species in a sample is determine using Beer's Law:

A=εbc

wherein: A is absorbance, b is pathlength of light through the sample, c is concentration and ε is the molar absorptivity of the sample. Using Beer's law and definitions of transmission and absorbance, the following relationship between concentration and transmission can be derived:

c=1/εb log 1/T

Where T is % transmission.

For highly absorbing solids, it is more desirable that the spectroscopic experiment be performed in the reflection mode, which indirectly represents absorption. In this mode, the sample is illuminated with light and reflected light is collected. There are two major types of reflection: specular and diffuse. Specular reflection contains no chemical information and is caused by the surface reflection of light. Reflection from mirrors and shiny surfaces are specular reflections. Diffuse reflection occurs after light has penetrated the sample, interacted with the material, then exits the sample surface. Diffuse reflectance contains both physical and chemical information. Diff-use reflectance spectroscopy can be thought of as an extension, using instrumentation, of human vision.

The quantification of diffuse reflectance is not as straight forward as is transmission. In a solid sample, the scattering of light causes the path length of light to be unknown. Thus, an unknown is left in Beer's law. The physics of diffuse reflection are very complicated, and not fully understood for all samples. However, the concept of using the ratio of Iₒ (intensity from the source) to Iᵣ (intensity at detector) can be used, even though a rigorous, functional relationship, between c and log (1/R) has not been developed. Concentrations in solids can be estimated using the following relationship where R is similar to T:

c∝log 1/R

Wherein R is Iᵣ/Iₒ

Herein, the term “wavelength” and the term “wavenumber” are alternatively used to characterize the specific infrared radiation being used or evaluated. In general, wavenumber is equal to 1 divided by the wavelength, in centimeters. Thus, for example, radiation of wavelength 750 nm has a wavenumber of 13,333 cm⁻¹.

In a typical Near Infrared (NIR) study, a sample is subjected to infrared light varied in the wavelength within a selected range between about 750 nanometers (nm) to 2500 nm (or 13,333 to 4,000 cm⁻¹ wavenumbers), with the resulting spectra plotted to show the amount of transmission through the sample (or diffusion reflectance), and thus the amount of infrared absorption by the sample.

As indicated, in some instances instead of studying the entire NIR range, only a selected portion of the NIR range is evaluated. The selection of the NIR range for evaluation can be made by first conducting an FTIR study of the sample, to determine a wavelength in the infrared indicative of differences between the samples being compared with respect to absorption fundamentals, and then calculating the appropriate overtone(s) for those identified wavelengths in the NIR, and basing the NIR data collection on those calculated overtones. The selection of the NIR range could alternatively be based on previous experience with the sample type; or by selection of the entire NIR range for which the NIR instrument is capable of data collection.

With respect to conduct of an IR study to identify absorption fundamentals, it is also noted that Fourier transform infrared techniques (FTIR) are well known for the development of a reliable bulk infrared spectra of samples. In general, FTIR techniques involve multiple infrared (IR) data collection from a single sample, in order to enhance evaluation of statistically meaningful absorption or transmission patterns, relative to instrument background noise.

The selection of near infrared (NIR) as an approach to collecting data for subjects of the type characterized herein with respect to food and/or agricultural materials is supported by the following:

1. Food and agricultural materials generally include chemical moieties which are active in the near infrared, i.e., which do exhibit significant absorption overtones in the near infrared. Such moieties include, for example, carbon-oxygen moieties, carbon-nitrogen moieties and oxygen-hydrogen moieties.

2. Near Infrared spectroscopic techniques currently available allow for:

(a) Evaluations across the near infrared taken at intervals (spacing) as little as about 2 to 3 nanometers (36 to 54 wavenumbers) throughout the NIR region;

(b) An ability to focus, for collection of data, over a substrate region of about 25 to 900 sq. mm with a spectral resolution of (typically) 2 nm; and

(c) A sensitivity of 2 to 3 times background noise, for many instruments.

In general, problems would have been expected with trying to utilize a near infrared study as an approach to conducting specific chemical distribution mapping with respect to agricultural and/or food materials because of expected difficulties with identifying a particular chemical moiety distribution (or component distribution) with respect to a complex background which also exhibits significant infrared activity.

In general, then, the issues of chemical imaging utilizing NIR analyses, for the agricultural and food industries, concern management of these and related issues.

II. NIR Chemical Imaging

In general, in accord with processes as described herein, a chemical morphology NIR-based image contrast plot is made of a selected sample. The term “chemical morphology NIR-based image contrast plot” is meant to refer to a plot showing different NIR absorption over a selected region of a sample. For microscopic imaging, the selected region is typically at least 15 by 15 microns in size, and also typically not greater than 3 mm by 3 mm, usually no more than about 500 by 500 microns in size. For macroscopic imaging, typically the region is smaller than 5 cm by 5 cm, for example, typically smaller than about 3 cm by about 3 cm. Techniques for generating such plots involve practical applications of chemometrics.

### A. Chemometrics generally

In general, the collection and processing of NIR data to accomplish the types of analyses and comparisons characterized herein, requires applications of various techniques sometimes characterized as “chemometrics.” Companies which specialize in chemometrics have been organized. For example, the NIR data collection and management techniques described herein, and equipment necessary to conduct the NIR data collection and management techniques, have been practiced by ChemIcon, Inc. of Pittsburgh, Pa. 15208. Another company which provides equipment and software necessary for performing an NIR imaging analysis is Spectral Dimensions, Olney, Md., 20832.

In general, the chemometrics techniques preferable in order to manipulate the NIR data collected in application of techniques according to the present invention, typically involve applications of one or more of three general areas of NIR data manipulation:

1. Preliminary data reduction;

2. Data analysis; and,

3. Prediction.

In general, data reduction is the technique of breaking down collected data into mathematically significant data variations. Data analysis generally involves data space dimensionality reduction and visualization techniques, conducted on data after more general (or preliminary) data reduction techniques have been applied.

The general principles and applications of data reduction will be understood by considering data reduction steps of the type that could be done to accomplish the comparison (FIGS. 5 and 6) characterized above for Example 1.

The NIR study of Example 1 concerns the evaluation of two paper samples, each of which is about 60 microns thick. One paper sample was made according to a standard wet laid technique, with only a standard component (wood cellulose fibers). The second paper sample was made similarly to the first sample, except for the addition of an agricultural material to be evaluated, namely, a particular seed based fiber additive. (The additive was provided in an amount of 1% by wt. based on total weight of solids in the paper-making slurry.) A purpose of the NIR study was to provide a chemical image (chemical morphology NIR-based image contrast plot) which indicates the distribution of the chemical variation provided by the agricultural additive (i.e., the seed based fiber additive), in the paper of the second sample.

In general, a first phase of the study was to determine whether the IR spectra of the two samples would indicate at least one wavelength region with overtones in the NIR by which the first and second samples could be differentiated from one another. In general, this can be evaluated utilizing bulk spectra obtained from a standard IR spectrometer, with standard IR techniques, for example FTIR.

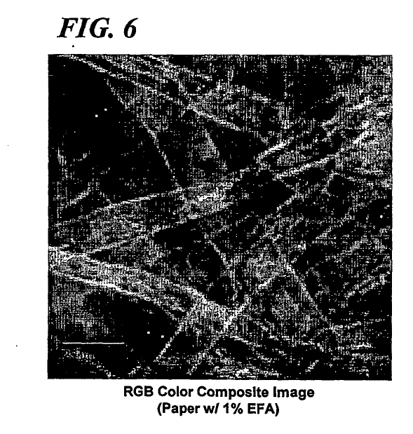

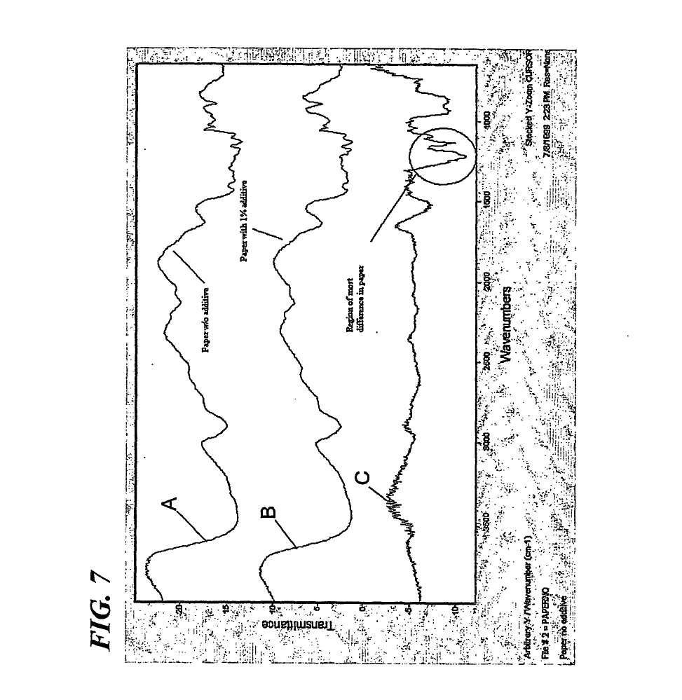

The samples, for example, were mounted in a standard IR spectrometer, in a standard manner, and the spectra were compared by computer. In FIG. 7, the two IR (FTIR) spectra are shown, the spectra at A of FIG. 7 being the paper without the additive, the spectra at B of FIG. 7 being the paper with the additive. A comparison of the spectra (A and B) shows that indeed differences can be determined, especially in the wavenumber region between 1,100 cm.⁻¹ and 1,300 cm.⁻¹ (i.e., 9090 to 7692 nm), specifically around peaks centered at 1,137 cm.⁻¹ (8795 nm) and 1,220 cm.⁻¹ (8196 nm). In FIG. 7, the plot shown at C is of the differences between spectra A and B.

Again, the purpose of the initial IR study to obtain bulk spectra, was to determine whether for the first and second samples to be evaluated, there could be identified at least one wavelength (or wavenumber) region in which they show a significant IR absorption difference from one another. The study as thus far described indicates that there was at least one wavelength (wavenumber) region in the IR where there were observable differences.

The differences observed in the fundamental absorptions of the FTIR can be used to calculate regions of overtones in the NIR which would also show differences. This is done by dividing the IR (FTIR) measured wavelength for the region of interest by whole integers since the overtones are found at such spacing. The overtones selected for data collection will be the one(s) falling within the NIR wavelengths. If the NIR wavelength sampling selected is based on such an overtone calculation, it will typically be convenient at least to collect data from a selected wavelength below the calculated overtone (for example 50 nm or 100 nm below) to a wavelength above the calculated overtone (for example 50 nm or 100 nm above), i.e., typically over a range of no more than 200 nm, or no more than 100 nm.

The next issue was whether or not those differences were statistically significant. In general, in this context the differences in NIR absorption (or transmission) are considered “significant”, if they are statistically meaningful with respect to the type of data collection and management techniques being conducted. That is, they will be “significant”, if the differences are ones which can be detected within the microscopic NIR techniques to be applied in the second phase of the study.

In general, an IR absorption (or transmission) measurement detected is of significance if the amount of instrumentation signal associated with it is greater than about three times the noise level of the instrumentation in the same region. Since the absorption bands in the NIR region of the spectrum arise from overtones of the IRs, calculations can be made to show that the expected differences in the NIR spectra between the first and second samples should be detectable given the differences observed in the FTIR.

The next phase or step, in general, involves collection of appropriate NIR data for the comparative analysis and generation of the chemical morphology NIR-based image contrast plot. For the comparative study, data needs to be collected, which can then be reduced for the chemical imaging. In general, the imaging data will be taken over a wavelength range selected from within the NIR range, for example from within about 1000 nm to 1700 nm, (or 10,000 to 5882 cm⁻¹) by the NIR instrument. Typical currently available instrumentation would have a region of less than 1000 microns square, (1 million sq. microns) and typically less than 500 microns square; for example about 330 microns square (108,900 sq. microns) defined by an instrumentation collector pixel organization of about 240 by 320, with each pixel oriented for collection of data from a sample area of about 1-2 square micron.

It is noted that the NIR region generally extends, as indicated above, from about 750 nm to 2500 nm. The characterization of collecting data within the region of about 1000 nm to 1700 nm was made due to the fact that currently available instrumentation, for example at ChemIcon, Inc., is configured to collect NIR data from this region. In some instances, the data collection will take place over the entire NIR region for which the equipment used is capable of collection. In others, selected wavelength regions within this range, for example based on calculated or observed overtones, will be used.

For an NIR study of the type characterized herein, chemical features (absorption differences) on the order of about 1 micron across, up to a couple hundred microns across, can be resolved and be shown in contrast. However, if a feature is much larger, a several hundred micron square field of view would not be large enough to generate an image of the feature; and, if a feature (region for contrast) is much smaller than about a micron, it would be below resolution currently expected with currently available equipment.

In general, this phase of the process concerns collection of a series of absorption data for each sample, within the NIR wavelength region determined, for example from the bulk IR spectra study. Typically, equipment is capable of sampling at about every 2 or 3 nm, (or 36 to 54 cm⁻¹ wavenumber) within the selected NIR range. Typical samplings would be at a fixed space typically selected from range of 5 nm to 10 nm inclusive (or 90 to 180 cm⁻¹ wavenumber), with the total number of data sets collected typically being at least 30 to 50 for each sample.

If, for example, the data collection was being done between 1000 nm and 1700 nm at every 5 nm, each sample would have approximately 141 spectra taken at each pixel in the 240 by 320 pixel region. Alternately stated, for the example being characterized, each pixel represents data collection over a sample area of about 1-2 square microns, if a 240 by 320 pixel pattern (76,800 pixels) is used to collect data from the NIR absorption of a 330 micron square sample exposure area. Thus, each sample would have been imaged by 76,800 different spectra, i.e., at 76,800 different locations, for each wavelength evaluated; with 141 different wavelengths evaluated. Of course, in some instances, if as many as 141 data sets, at 141 different wavelengths, are collected, it may be decided, during data reduction, to exclude certain of the wavelengths from the data workup, to facilitate data reduction. The excluded wavelengths could be, for example, wavelengths not expected to show significant differences for the samples being compared, either by observation or based on calculated overtones from the FTIR study.

It is noted that for the studies done to generate the images of FIGS. 5 and 6, NIR data was collected over the region of 1000 to 1700 nm, at every 5 nm.

Once the NIR spectral data has been collected, a data reduction process is initiated, to break down this data into mathematically significant data variations.

Initially, a number of effects need to be cancelled out. For example, there may be data collection variations due to light intensity variations, optical throughput variations, etc., i.e., equipment variations, in the area of the data collection or over the time period in which the data collection occurred. In general, these variations are managed out of the data set by using a material or standard for which the absorption is fully understood, and for which it is homogenous, and then evaluating the standard sample through the same wavelengths and with the same machine settings. In general, the reference would be selected to recreate variances in the system other than those from the chemistry or morphology of the paper samples to be evaluated. For example, if during evaluation each of the paper samples was mounted on a blank microscope slide, the study to manage the machine variations, etc., would be generated by using the microscope slide as the standard, but without a sample mounted on it. In general, the reduction of the data for the two paper samples would occur by taking the image for each sample to be evaluated and dividing it by the reference image.

At this point, the two sets of data for the two different paper samples would indicate NIR absorption variations from the paper samples themselves, and not from the variables or issues of the NIR system itself. However, the data would still contain a component which results from variations in the physical morphology of the paper, as opposed to merely chemical variations.

For example, if one considers the cellulose fiber paper sample not having the additive therein, variations in absorption (at different regions) of that sample could be the result of differences in the amount of cellulose fiber present in the path of the NIR beam, as opposed to variations in the actual chemistry of material in the path of the NIR beam. This would result, for example, by having one pixel collecting data from an NIR beam that passed through one pulp fiber, and another pixel measuring NIR beam that passed through five or six stacked pulp fibers. The absorption differences would reflect path length or thickness variations, as opposed to actual chemical distribution variations from the pulp fibers.

Another issue with respect to this would be focal point or focal plane selection; that is, variations regarding precisely were the point of the focus of the instrument occurs in the sample.

Once the two data sets have been reduced to a point where the data reflects NIR absorption variations from the paper itself, and not equipment or instrumentation variations, generally the next step of data reduction or pre-processing is conducted by a statistical technique generally referred to as vector normalization or data normalization. The result of this normalization is that the variations resulting from the path length variations, focal plane variations or similar variations are factored out of the data. In general, vector normalization techniques involve dividing each spectrum of the image by its vector norm.

As indicated above, another variable managed in the normalization, is a variable resulting from depth of focus. The samples of Example 1, for example, each had a 60 micron thickness. For an NIR set taken with a 20× objective, the depth of focus would be about ten microns. Indeed, particularly for a sample of about 60 microns thick, one would choose a depth of focus on the order of 5 to 12 microns. The normalization conducted, would factor out intensity differences due to this focal length variation.

After vector normalization, the NIR data would now only exhibit (statistically meaningful) differences resulting from the pattern of absorption, and not the amount of absorption. That is, for example, the data would now show a constant (homogenous) chemical composition, if the only material in the path of the infrared beam, for a selected pixel, was cellulose fiber, without regard to variations in the cellulose fiber thickness or distribution.

The next portion of a typical study would be to plot the data and observe contrasts which can be attributable to chemical composition differences resulting from the fiber additive. That is, the next step is preparation of the actual chemical morphology NIR-based image contrast plot. In some instances, this plotting can be done by conducting a further data reduction or manipulation technique, generally referred to as principal component analysis PCA).

A Principal Component Analysis (PCA) is a data space dimensionality technique. In general, a least squares line is drawn through the maximum variance in the n-dimensional data set. The vector resulting from the least squares fit is termed the first principal component (PC1) for the first loading; the term “loading” referring to the eigenvectors that result from a singular value decomposition of the covariance and matrix of the original data. After subtracting the variance explained from the PC1, the operation is repeated and a second principal component (PC2) is calculated. This process can be repeated until some percentage of the total variance of the data space is explained (normally 95% or greater). Individual PC score images can then be visualized to reveal or image information including sample information, as well as instrument response, including noise; the term “score” referring to projection of the original data upon the loadings. Reconstruction of spectral dimension data can then be performed by cluster analysis, including PC's that described material or instrument parameters that one desires to amplify or suppress, depending on the needs of the sensing application. Each loading would be a separate chemical morphology NIR-based image contrast plot.

Again, Principal Component Analysis is a standard data reduction technique. Instrumentation and software performing such techniques are available commercially from such sources as ChemIcon, Inc. of Pittsburgh, Pa. 15208; and, organizations such as ChemIcon, Inc. can also be retained to perform such analyses.

In general, as one plots selected loadings, (PC1, PC2, PC3, etc.), based on knowledge of how the sample was prepared, one can identify the loading which most meaningfully indicates the chemical image or distribution of interest. For example, considering the paper coating example being discussed as an example and described in Example 1 below, an investigator would know that: in general, the paper samples each substantially comprise (greater than 95% by wt.) large cellulose fibers, visible for example in the SEM; and, that the material added to the paper is a dimensionally fine seed based fiber. When reviewing that various loading plots (from the PCs generated by PCA) once the comparison represented at PC3 was observed, it should be understood that this principal component (score image and loading plot) represents the chemical image or chemical distribution image being sought; i.e., the distribution of the additive in the paper. The reason for this is that with respect to this principal component (PC3); the paper sample PCA score image (composite image of the 2nd, 3rd, and 4th PC score images) without the additive is shown to be homogenous; and the analogous PCA score image of the paper with the additive shows the distribution of the additive in a form which makes sense, i.e., defining (or surrounding) the large cellulose fibers of the paper. This PC composite image indicates, then, that the additive was primarily distributed along the cellulose (pulp) fibers as a form of coating, as opposed to between the fibers or in some other spatial relation.

In addition to analyzing the chemical and physical components of the current sample image, data analysis methods such as PCA can be used to predict such components in future images taken on similar samples. In general, this is done by first establishing a standard PCA model using a sample of known properties. If it is not possible to obtain such a standard sample, the user may define one. For relative predictions, this user-defined standard can be arbitrary: for example, the “average” sample in a collection. Then, when a sample of similar but unknown properties is imaged, the standard sample's PCA-pretreatment scaling parameters are applied to this new sample. The scaled data is then projected on the loadings of the standard sample's PCA model, in order to generate scores images that illustrate the properties of the new sample relative to the standard one. Also, an inspection of the residual statistics images that determine how well the standard model “fits” the new sample, will reveal whether the new sample contains components that are not present in the standard. Finally, there are several extensions of this method, generally known as “cluster analysis” methods, which use slightly different means to accomplish the same objective of component prediction. One such method is called SIMCA (Soft Independent Method of Class Analogy modeling), which actually builds separate PCA models for each user-defined type of component or “class” in the standard sample image, and then projects future sample images onto each class model in order to determine the amount and type of components in the new sample image.

In addition to PCA, more conventional spectroscopic analysis methods can be used to classify new samples relative to a standard. For example, if the pure spectrum or spectra of the component of interest is known, then spectra from unknown sample images can be projected onto the standard spectrum (i.e., by scalar multiplication of the normalized spectral vectors) to generate a similarity value that, when imaged, will show how closely the new sample matches the old one. Note that even if a pure standard spectrum is not known, methods related to PCA (collectively known as Multivariate Curve Resolution or MCR) can be used to determine pure component spectra from a given image. If one wishes to predict not the type of components in a new image, but the amount of a certain component relative to a standard, this can be done using a simple ratio of the properly scaled spectral feature between the unknown sample image and the standard. Examining the relative magnitudes in the scores images of the standard PCA model and the new sample's projection onto that model will also accomplish this.

III. Applications of Use; General Procedures

### A. Agricultural and/or food materials

As indicated above, the present disclosure is concerned with evaluations concerning agricultural and/or food materials. In general, the term “agricultural and/or food materials” and variants thereof, in this context, is meant to exclude applications involving human medical and pharmaceutical studies.

Herein the term “agricultural material” is meant to refer to a material derived from an agricultural industry source. In general, the term is meant to refer to a material which, therefore, is derived from either a plant source or an animal source. The term “derived” in this context is meant to indicate that the agricultural material or additive may be obtained directly, in whole or in part, from a plant or animal source, or may be the result of processing material (by either chemical or medical modification) which was derived from a plant or animal source. The fiber additive discussed in Example 1 is an example of an agricultural material. It was derived from a plant source, i.e., seed-based fiber. It was obtained from the plant source, plant seeds, by processing as characterized, and thus is not in its natural form.

In general, a characteristic of agricultural materials is that they will typically comprise organic materials, and thus are generally primarily compounds of carbon, hydrogen, oxygen and sometimes nitrogen. In typical instances, the chemical makeup for the agricultural materials will be relatively complex organic substances; for example, carbohydrates, lipids and proteins. Typical substances formed from such materials would include: cellulose(s), hemicellulose(s); lignin(s); starch(es); dextrin(s); dextran(s) polysaccharide(s); collagen(s); elastin(s); gelatin(s); triglyceride(s); fatty acid(s); amino acid(s); starch hydrolysate(s); and combinations, modifications or derivatives of these materials. In many instances, the material will include water.

Herein the term “food material” and variants thereof is meant to refer to a food material derived from or used in a food industry, again typically from a plant or animal source. In general, food materials are derived from the same sources (or groups of materials or compounds) as identified above with respect to the term “agricultural material,” and comprise similar chemical materials.

Herein in some instances, a material will be referred to as an “additive.” In general, an additive is a material which is added as an ingredient in a material, composition or substrate as opposed to one formed in the composition or substrate. In general, an additive would be a material which, with respect to the total weight of the composition or substrate (after addition), comprises no greater than 50% by weight. As indicated above, techniques according to the present invention can, in some selected applications, detect and map (i.e., image) the presence of an additive, in a composition in which the additive is present at no greater than 10% by weight and often no more than 2% by wt., indeed, for example in amounts on the order of 0.1-1.5% by weight.

Herein, the term “component” is meant to refer to an identifiable material, anomaly, variation, etc., within a composition. An “additive” would thus be a “component” of an overall composition. Components would include, for example, distributed materials which are formed within the composition, during formation of the composition or later, for example, as crystals, etc.

### B. Examples of Studies

The following list provides a non-exclusive (or non-exhaustive) set of examples of the types of studies that can be conducted with techniques as characterized herein:

1. Distribution of selected proteins, for example in flours;

2. Carbohydrate distributions, for example in marshmallows;

3. Evaluation of crystal distribution, for example in gelatins; as a specific example, crystal distributions in gummi bears;

4. Evaluation of crystal formation and distribution, for example, in ice creams;

5. Evaluation of starch coating distributions, for example in hard candies;

6. Evaluation of fat types and distributions, for example in chocolate or meat;

7. Evaluation of protein and starch distributions, for example, in tortillas;

8. Evaluation of protein, oil, water or carbohydrate distribution, for example in grains;

9. Evaluation of digestion processes, through measuring food changes in time, as the food passes through the digestive process of an animal; and

10. Evaluating a malting process and the changes which occur within the material.

### C. Products from studies according to the techniques described herein

Techniques applied according to the present invention can be utilized to generate such products as chemical morphology NIR-based image contrast plots of: agricultural and/or food substrates or compositions; or agricultural and/or food material additions into a composition or substrate. An example of such a plot is illustrated in FIG. 6, i.e., the PC score composite image (or plot) depicted.

Such plots, are, in general, products of selected processes according to the present disclosure.

### D. Products characterized according to the techniques described herein

In general, products can be characterized in accord with the techniques described herein. An example would be the paper material of Example 1, with the additive. When characterized according to the techniques described herein, in general, the paper sample could be characterized as possessing, distributed on the cellulose fibers, a chemical distribution which indicates a statistically significant absorption difference, from background cellulose, in the 1100 cm⁻¹-1300 cm⁻¹ wavenumber region of the IR and the corresponding overtones in the NIR. The additive could be characterized as being an additive which produces such an effect.

### E. General Process Description

From the information provided herein, general processes and techniques in accord with the current teachings become apparent. In general, the techniques involve: (1) a process of evaluating distribution (or effect) of an agricultural or food component in a composition; or (2) a process of evaluating distribution (or effect) of a component in an agricultural or food composition. The process generally includes steps of:

(a) Obtaining a first sample for evaluation;

(b) Selecting at least one NIR wavelength (wavenumber) region indicative of the component as distinguished from the remainder of the composition;

(c) Preparing a chemical morphology NIR-based image contrast plot, for example based on the at least one NIR wavelength (wavenumber) region selected in step (b), to depict distribution of the selected component in the composition; and,

(d) Evaluating the chemical morphology NIR-based image contrast plot, to discern distribution of the chemical moiety or chemical characteristic plotted.

The step (b) of selecting at least one NIR wavelength or wavenumber region indicative to the component as distinguished from a remainder of the composition, may be based upon conducting an IR (for example an FTIR study) showing fundamental absorptions, and then calculating the appropriate overtones in the NIR for data collection. However, such an FTIR study is not required. For example, the step of selecting at least one NIR wavelength region could be made by deciding to conduct a study throughout the entire or a selected portion of the NIR region, or by selecting a region of the NIR based upon previously obtained data or information.

In many applications, the process will in general involve evaluating distribution of an agricultural food component in an agricultural or food composition. However, the techniques herein are not limited to instances in which both materials (the component and the remainder of the composition) are agricultural or food materials.

In typical applications, the component to be evaluated, will comprise no more than 50%, by wt., of the sample. In typical microscopic applications, the chemical morphology NIR-based image contrast plot will be selected to depict a region of the sample covering not more than one million square microns, and typically not more than 0.5 million square microns. In typical preferred microscopic applications, the region will be about 19,000 to 27,000 square microns. In typical macroscopic evaluations, the region imaged will be no more than 50 cm by 50 cm (2500 sq. cm), typically no more than 5 cm by 5 cm (25 sq. cm), most typically 3 cm by 3 cm (9 sq. cm) or smaller.

In typical applications, the sample will be selected to have a thickness not greater than 100 microns, often within the range of about 10 to 60 microns.

In many applications, the component to be evaluated will comprise an additive provided in the composition; again, often at no more than about 50% by wt., and indeed often not more than about 10% by wt., for example at 2% or less and in some applications no more than 1.5% by wt., for example 0.1%-1.5% by wt. In many typical instances, the additive will comprise an agricultural or food component comprising at least 50%, typically at least 80%, often at least 90% by wt., material selected from the group consisting essentially of: carbohydrates, lipids, proteins and mixtures thereof.

In typical applications, the composition, other than the additive, can also be characterized as selected from agricultural or food material, comprising at least 50% by wt., (typically at least 80%, often at least 90% by wt.) material selected from the group consisting essentially of: carbohydrates, lipids, proteins and mixtures thereof.

Typical agricultural or food materials useable as either the component or the overall composition are materials comprising (except for H₂O) at least 70%, often at least 80%, typically at least 90%, by wt., material selected from the group consisting essentially of: cellulose(s), hemicellulose(s), lignin(s), starch(es), dextrin(s), dextran(s), polysaccharide(s), collagen(s), elastin(s), gelatin(s), triglyceride(s), fatty acid(s), amino acid(s), starch hydrolyzate(s) and combinations, modifications or derivatives of these materials. A similar definition is useable for material in a composition which is not the component to be evaluated.

In some applications, the process will involve obtaining first and second samples, and evaluating them as comparatives. In general, for example, the first sample would include the component to be evaluated; and, the second sample would be a comparative example. A typical comparative example would comprise the same composition, but without the component. In some instances, the comparative example could contain a known amount of the component or a known variation.

In typical comparative studies, it may be useful to plot a principal component in which one of the samples shows substantially homogenous chemical morphology, while the other sample shows non-homogenous chemical morphology. Typically, when such is the case, the sample showing the non-homogenous chemical morphology will be the one which includes the component to be mapped, and the sample showing substantially homogenous chemical morphology will be the comparative.

As indicated by the Example 1 described below, for studies involving materials similar to wood pulp and a seed based fiber additive, the chemical morphology NIR-based image contrast plots can be selected based upon differences observable in bulk FTIR spectra within a wavenumber region selected from the range of 1100 cm⁻¹-1300 cm⁻¹, inclusive.

IV. A General Characterization of Equipment and Methods for Conducting a Chemical Imaging Investigation

As indicated above, the equipment, data management software and techniques necessary for conduct of chemical imaging investigation in accord with the principles described herein, are available from suppliers of analytical equipment, data handling software and computer control systems and programming. In addition, the NIR data gathering techniques, and data reduction, can generally be outsourced, i.e., be provided to an investigator by an analytical supply/service company. One such company is Chemicon, Inc. of Pittsburgh, Pa. 15208.

Chemicon, Inc. has provided a general description of useable equipment and techniques in its U.S. provisional application Ser. No. 60/239,969, filed on Oct. 13, 2000, which describes application of near infrared spectroscopic technique for automatically inspecting certain inorganic inclusions of interest to the semiconductor fabrication industry. The following discussion, in this section IV, is derived from the portion of the ChemIcon provisional disclosure relating to equipment and software identification. It is understood that Chemi-Con has filed an additional disclosure, namely, U.S. Pat. No. 09/976,391 entitled “Near Infrared Chemical Imaging Microscope.”

According to the ChemIcon provisional disclosure, NIR imaging can be conducted with a near infrared spectroscopic microscope apparatus employing NIR absorption molecular spectroscopy. The microscope and experiment design could, for example, involve: (1) using NIR optimized liquid crystal (LC) imaging spectrometer technology for wavelength selection; (2) using an NIR optimized refractive microscope in conjunction with infinity-corrected objectives to form the NIR image on the detector without the use of a tube lens; (3) an integrated parfocal analog color CCD detector real-time sample positioning and focusing; (4) appropriate means for fusing the color image and the NIR image in software; (5) the use of the NIR microscope as a volumetric imaging instrument through the means of moving a sample through focus, collecting image in and out of focus and reconstructing a volumetric image of the sample in software, or through the means of keeping the sample fixed and changing the wavelength dependent depth of penetration in conjunction with a refractive tube lens with a well characterized chromatic effect; (6) coupling the output of the microscope to an NIR spectrometer either via direct optical coupling or via a fiberoptic; (7) utilization of seeding approaches to seed a sample material of known composition, structure and/or concentration, and then generating the NIR image suitable for qualitative and quantitative analyses; and (8) means for analyzing and visualizing the NIR chemical images using chemical image analysis software. Such technology can be readily applied on a microscope optic platform.

In addition to utilizing standard optical microscope hardware described in the ChemIcon provisional disclosure, NIR hyperspectral imaging may be performed using properly configured optical systems designed for imaging in the macroscopic domain. This domain, as defined in this disclosure, extends from above an approximately 3 mm by 3 mm field of view up to an approximate field of view of 50 cm by 50 cm. The imaging system and experiment design could, for example, involve: (1) using NIR optimized liquid crystal (LC) imaging spectrometer technology for wavelength selection; (2) using a suitable NIR illumination system that will uniformly illuminate a sample of interest at the object plane of a suitable optical system with sufficient intensity and wavelength band pass for acquiring NIR hyperspectral images; (3) appropriate mounting hardware for the LCTF/Camera system which allows for precise positioning of the imaging system relative to the sample of interest; (4) appropriate high resolution sample translation stage (achieving translational and rotational movement along 3 linear and up to three rotational axes) for fine focus and region of interest location; (5) using an NIR optimized imaging lens having an industry standard image format size and a suitably designed transfer optic to transform the image format at the focal plane of the lens to an infinity corrected image at the detector plane (OR) a suitable infinity corrected NIR optimized inspection objective capable of imaging regions considered to be of macroscopic scale; (6) an integrated parfocal analog color CCD detector real-time sample positioning and focusing; (7) appropriate means for fusing the color image and the NIR image in software; (8) the use of the NIR microscope as a volumetric imaging instrument through the means of moving a sample through focus, collecting image in and out of focus and reconstructing a volumetric image of the sample in software, or through the means of keeping the sample fixed and changing the wavelength dependent depth of penetration in conjunction with a refractive tube lens with a well characterized chromatic effect; (9) coupling the output of the microscope to an NIR spectrometer either via direct optical coupling or via a fiberoptic; (10) utilization of seeding approaches to seed a sample material of known composition, structure and/or concentration, and then generating the NIR image suitable for qualitative and quantitative analyses; and 11) means for analyzing and visualizing the NIR chemical images using chemical image analysis software. Such technology can be readily applied on a microscope optic platform.

In the approaches described in the ChemIcon provisional disclosure, an NIR optimized liquid crystal (LC) imaging spectrometer technology is used for wavelength selection; and, it is indicated that the LC imaging spectrometer may be of the following types: Lyot liquid crystal tunable filter (LCTF); Evans Split-Element LCTF, Solc LCTF; Ferroelectric LCTF; Liquid crystal Fabry Perot (LCFP); or a hybrid filter technology resulting from a combination of the above-mentioned LC filter types or the above mentioned filter types in combination with fixed bandbass and bandreject filters comprised of dielectric, rugate, holographic, color absorption, acousto-optic or polarization types.

According to the ChemIcon provisional disclosure, an NIR optimized refractive microscope can be employed in conjunction with infinity-corrected objectives to form the NIR image on the detector without the use of a tube lens. The microscope can be optimized for NIR operation through inherent design of objective and associated anti-reflective coatings, condenser and light source. To simultaneously provide high numerical apertures, the objective should be refractive. To minimize chromatic aberration, maximize throughput and reduce cost the conventional tube lens can be eliminated, while having the NIR objective form the NIR image directly onto the NIR focal plane array (FPA) detector, typically of the InGaAs type. The FPA can also be comprised of Si, SiGe, PtSi, InSb, HgCdTe, PdSi, Ge, or analog vidicon types. The FPA output can be digitized using a frame grabber approach.

According to the ChemIcon provisional disclosure, one can use an integrated parfocal analog CCD detector for real-time sample positioning and focusing. An analog video camera sensitive to visible radiation, typically a color or monochrome CCD detector, but which, may be comprised of a CM.OS type, would be positioned parfocal with the NIR FPA detector to facilitate sample positioning and focusing without requiring direct viewing of the sample through convention eyepieces. The video camera output could be digitized using a frame grabber approach.

According to the ChemIcon provisional disclosure, one can use a means for fusing the color image and the NIR image in software. While the NIR and visible cameras often generate images having differing contrast, the sample fields of view can be matched through a combination of optical and software manipulations. As a result, the NIR and visible images can be compared and even fused through the use of overlay techniques and correlation techniques to provide the user a near-real time view of both detector outputs on the same computer display. The comparative and integrated views of the sample can significantly enhance the understanding of sample morphology and architecture. By comparing the visible, NIR and NIR chemical images, additional useful information can be acquired about the chemical composition, structure and concentration of species in samples.

Also according to the ChemIcon provisional disclosure, one can use the NIR microscope as a volumetric imaging instrument through the means of moving the sample through focus, collecting images in and out of focus and reconstructing a volumetric image of the sample in software. For samples having some volume (bulk materials, surfaces, interfaces, interphases), volumetric chemical imaging in the NIR can be useful for failure analysis, product development and routine quality monitoring. The potential also exists for performing quantitative analysis simultaneous with volumetric analysis. Volumetric imaging can be performed in a non-contact mode without modifying the sample through the use of numerical confocal techniques, which require that the sample be imaged at discrete focal planes. The resulting images are processed and reconstructed and visualized. An alternative to sample positioning is to employ a tube lens in the microscope which introduces chromatic aberration. As a result the sample can be interrogated as a function of sample depth by exercising the LC imaging spectrometer, collecting images at different wavelengths which penetrate to differing degrees into bulk materials. These wavelength dependent, depth dependent images can be reconstructed to form volumetric images of materials without requiring the sample to be moved.

It is stated in the ChemIcon provisional disclosure that the imaging process can couple the output of the microscope to a NIR spectrometer either via direct optical coupling or via a fiber optic and that this allows conventional spectroscopic tools to be used to gather NIR spectra for traditional, high speed spectral analysis. The spectrometers can be of the following types: fixed filter spectrometers; grating based spectrometers; Fourier Transform spectrometers; or Acousto-Optic spectrometers.

A chemical imaging addition method involves seeding the sample with a material of known composition, structure and/or concentration and then generating the NIR image suitable for qualitative and quantitative analysis. A practice would be to construct a standard calibration curve which is a plot of analytical response for a particular technique as a function of known analyte concentration. By measuring the analytical response from an unknown sample, an estimate of the analyte concentration can then be extrapolated from the calibration curve. With this method known quantities of the analyte (additive) are added to the samples and the increase in analytical response is measured. When the analytical response is linearly related to concentration, the concentration of the unknown analyte can be found by plotting the analytical response from a series of standards and extrapolating the unknown concentration from the curve. In this graph, however, the x-axis is the concentration of added analyte after being mixed with the sample. The x-intercept of the curve is the concentration of the unknown following dilution. The primary advantage of this method is that the matrix remains constant for all samples.

The chemical imaging addition method can be used for both qualitative and quantitative analysis. The chemical imaging addition method relies upon spatially isolating analyte standards in order to calibrate the chemical imaging analysis. In chemical imaging, thousands of linearly independent, spatially-resolved spectra are collected in parallel or analytes found within complex host matrices. These spectra can then be processed to generate unique contrast intrinsic to analyte species without the use of stains, dyes, or contrast agents. Various spectroscopic methods including near-infrared (NIR) absorption spectroscopy can be used to probe molecular composition and structure without being destructive to the sample. Similarly, an NIR chemical imaging the contrast that is generated reveals the spatial distribution of properties revealed in the underlying NIR spectra.

According to the ChemIcon provisional disclosure, the chemical imaging addition method can involve several data process steps, including:

1. Ratiometric correction, in which the sample NIR image is divided by the background NIR image to produce a result having a floating point data type.

2. Normalizing the divided image by dividing each intensity value at every pixel in the image by the vector norm for its corresponding pixel spectrum. Where the vector norm is the square root of the sum of the squares of pixel intensity values for each pixel spectrum.

3. Cosine correlation analysis (CCA) which is a multivariate image analysis technique that assesses similarity in spectral image data while simultaneously suppressing background effects. CCA assesses chemical heterogeneity without the need for training sets, identifies differences in spectral shape and efficiently provides chemical image based contrast that is independent of absolute intensity. The CCA algorithm treats each pixel spectrum as a projected vector in n-dimensional space, where n is the number of wavelengths sampled in the image. An orthonormal basis set of vectors is chosen as the set of reference vectors and the cosine of the angles between each pixel spectrum vector and the reference vectors are calculated. The intensity values displayed in the resulting CCA images are these cosine values, where a cosine value of 1 indicates the pixel spectrum and reference spectrum are identical, and a cosine value of 0 indicates the pixel spectrum and the reference spectrum are orthogonal (no correlation). The dimensions of the resulting CCA image is the same as the original image because the orthonormal basis set provides n reference vectors, resulting in n CCA images.

4. Principal component analysis (PCA), which is a data space dimensionality reduction technique. A least squares fit is drawn through the maximum variance in the n-dimensional dataset. The vector resulting from this least squares fit is termed the first principal component (PC) or the first loading. After subtracting the variance explained from the first PC, the operation is repeated and the second principal component is calculated. This process is repeated until some percentage of the total variance in the data space is explained (normally 95% or greater). PC Score images can then be visualized to reveal orthogonal information including sample information, as well as instrument response, including noise. Reconstruction of spectral dimension data can then be performed guided by cluster analysis, including without PCs that describe material or instrument parameters that one desires to amplify or suppress, depending on the needs of the sensing application.

According to the ChemIcon provisional disclosure, typical applications of the techniques described would involve analyzing and visualizing the NIR chemical images using chemical image analysis software and a comprehensive description of the variety of steps used to process chemical images is provided.

According to the ChemIcon provisional disclosure, until recently, seamless integration of spectral analysis, chemometric analysis and digital image analysis has not been commercially available. Individual communities have independently developed advanced software applicable to their specific requirements. For example, digital imaging software packages that treat single-frame gray-scale images and spectral processing programs that apply chemometric techniques have both reached a relatively mature state. One limitation to the development of chemical imaging, however, has been the lack of integrated software that combines enough of the features of each of these individual disciplines to have practical utility.

According to the ChemIcon provisional disclosure, historically practitioners of chemical imaging were forced to develop their own software routines to perform each of the key steps of the data analysis. Typically, routines were prototyped using packages that supported scripting capability, such as Matlab, (available from The Mathworks, Inc., Natick, Mass.) IDL (available from Research Systems, Boulder, Colo.), Grams (available from ThemoGalactic, Inc., Salem, N.H.) or LabView (available from National Instruments, Austin, Tex.). According to the ChemIcon disclosure, these packages, while flexible, are limited by steep learning curves, computational inefficiencies, and the need for individual practitioners to develop their own graphical user interface (GUI). Today, commercially available software does exist that provides efficient data processing and the ease of use of a simple GUI.

Software that meets these goals must address the entirety of the chemical imaging process. The chemical imaging analysis cycle illustrates the steps needed to successfully extract information from chemical images and to tap the full potential provided by chemical imaging systems. The cycle begins with the selection of sample measurement strategies and continues through to the presentation of a measurement solution. The first step is the collection of images. The related software must accommodate the full complement of chemical image acquisition configurations, including support of various spectroscopic techniques, the associated spectrometers and imaging detectors, and the sampling flexibility required by different sample sizes and collection times. Ideally, even relatively disparate instrument designs can have one intuitive GUI to facilitate ease of use and ease of adoption.

The second step in the analysis cycle is data preprocessing. In general, preprocessing steps attempt to minimize contributions from chemical imaging instrument response that are not related to variations in the chemical composition of the imaged sample. Some of the functionalities needed include: correction for detector response, including variations in detector quantum efficiency, bad detector pixels and cosmic events; variation in source illumination intensity across the sample; and gross differentiation between spectral line shapes based on baseline fitting and subtraction. Examples of tools available for preprocessing include ratiometric correction of detector pixel response; spectral operations such as Fournier filters and other spectral filters, normalization, mean centering, baseline correction, and smoothing; spatial operations such as cosmic filtering, low-pass filters, high-pass filters, and a number of other spatial filters.

Once instrument response has been suppressed, qualitative processing can be employed. Qualitative chemical image analysis attempts to address a simple question, “What is present and how is it distributed?” Many chemometric tools fall under this category. A partial list includes: correlation techniques such as cosine correlation and Euclidean distance correlation; classification techniques such as principal components analysis, cluster analysis, discriminate analysis, and multi-way analysis; and spectral deconvolution techniques such as SIMLISMA and multivariate curve resolution.

Quantitative analysis deals with the development of concentration map images. Just as in quantitative spectral analysis, a number of multivariate chemometric techniques can be used to build the calibration models. In applying quantitative chemical imaging, all of the challenges experienced in non-imaging spectral analysis are present in quantitative chemical imaging, such as the selection of the calibration set and the verification of the model. However, in chemical imaging additional challenges exist, such as variations in sample thickness and the variability of multiple detector elements, to name a few. Depending on the quality of the models developed, the results can range from semi-quantitative concentration maps to rigorous quantitative measurements.

Results obtained from preprocessing, qualitative analysis and quantitative analysis must be visualized. Software tools must provide scaling, automapping, pseudo-color image representation, surface maps, volumetric representation, and multiple modes of presentation such as single image frame views, montage views, and animation of multidimensional chemical images, as well as a variety of digital image analysis algorithms for look up table (LUT) manipulation and contrast enhancement.

Once digital chemical images have been generated, traditional digital image analysis can be applied. For example, Spatial Analysis and Chemical Image Measurement involve binarization of the high bit depth (typically 32 bits/pixel) chemical image using threshold and segmentation strategies. Once binary images have been generated, analysis tools can examine a number of image domain features such as size, location, alignment, shape factors, domain count, domain density, and classification of domains based on any of the selected features. Results of these calculations can be used develop key quantitative image parameters that can be used to characterize materials.

The final category of tools, Automated Image Processing, involves the automation of key steps or of the entire chemical image analysis process. For example, the detection of well-defined features in an image can be completely automated and the results of these automated analyses can be tabulated based on any number of criteria (particle size, shape, chemical composition, etc.). Automated chemical imaging platforms have been developed that can run for hours in an unsupervised fashion.

The ideal analysis package should support the user's efforts to carefully plan experiments and optimize instrument parameters and should allow the maximum amount of information to be extracted from chemical images so that the user can make intelligent decisions. ChemIcon has developed a chemical image software package, ChemImage, which supports many sophisticated analysis tools.

V. Experimental

### EXAMPLE 1

### NIR Imaging of Fiber Addition to Paper

The disclosure of this example is found in U.S. application Ser. No. 09/689,994, filed Oct. 13, 2000, and assigned to Cargill, Inc.; the complete disclosure of U.S. Ser. No. 09/689,994 being incorporated herein by reference.

### A. Preparation of the Agricultural Additive (modified seed based fiber).

Step 1. Acid Treatment of Fiber

Corn fiber (SBF-C) was obtained from Cargill Corn Milling, Cedar Rapids, Iowa. The corn fiber (SBF-C) was washed on a 70-mesh screen using a fine spray of water to remove fiber fines, free starch and protein. The moisture content of the resulting washed fiber was determined to be 50%. Approximately 1200 grams (600 grams on dry basis) of the fiber was then loaded in the screened basket (having a 100-mesh screened bottom) of an M/K digester and inserted in the pressure vessel.

A dilute acid solution containing 2% sulfuric acid (based on fiber dry weight) was combined with the SBF at a ratio of dilute acid solution to SBF of 10:1 (weight basis). The dilute acid solution contained 12 grams of 100% sulfuric acid (or 12.5 grams of the acid purchased at 96% concentration) and 5387.5 grams of water. The amount of sulfuric acid and water in the dilute acid solution was determined as shown below:

| | |
|---|---|
| Total weight of the | 600 g × 10 = 6000 g |
| dilute acid solution: | |
| Amount of water | 6000 − 600 g (from wet fiber) − 12.5 g of |
| needed: | H₂SO₄ = 5387.5 g of water |

The dilute acid solution was slowly added to the corn fiber in the digester and the circulation pump was turned on. After confirming that the dilute acid solution was being circulated in the reactor, the reactor lid was sealed. The reaction temperature was set at 120° C. and time to reach reaction temperature was set at 45 minutes and then was set to be maintained for 1 hour. The heater in the reaction vessel was turned on. The temperature and pressure inside the reactor were recorded as a function of time. After reaching the target temperature of 120° C., the reaction was continued for 1 hour. After 1 hour, the cooling water supply to the reactor was turned on to cool the reactor contents. The spent dilute acid solution was drained from the reactor by opening a drain valve on the reactor. The fiber content in the reactor basket was carefully removed and washed using two washing batches of 6 liters of water each. The washing was continued further until the wash water had a neutral pH (e.g., between 6.0 and 8.0, typically about 7.0).

Step 2. Acid Chlorite Treatment

The acid treated fiber from Step 1 was then treated in a surface modification step. The acid treated fiber was combined with an acid chlorite solution to form a fiber slurry that included 10% fiber and 90% acid chlorite solution. The acid chlorite solution included 1.5% by weight (based on dry fiber) of sodium chlorite and 0.6% by weight (of dry fiber) of hydrochloric acid. The reaction was carried out in a sealed plastic bag at a temperature of 65-75° C. for 1 hour at a pH between about 2 and 3. After treatment with the acid chlorite solution, the fiber slurry was diluted with 2 liters of water and filtered in a Buchner type funnel. This step was repeated until the resulting filtrate was clear and at neutral pH (e.g., pH 6.0 to 8.0, preferably about 7.0).

Step 3. Peroxide Treatment

The acid chlorite treated fibers from Step 2 were then treated with an alkaline peroxide solution. The fibers were combined with 3-8% by weight (of the dry fiber) of hydrogen peroxide and 2% by weight (of the dry fiber) sodium hydroxide at a pH between about 10-10.5 and at a solids concentration of 10-20%. Sodium metasilicate was added (3% by weight of dry fiber) as a chelating agent. The peroxide treatment step was conducted in a sealed plastic bag at 60-65° C. for 1 hour. After the reaction, the fiber slurry was diluted with 2 liters of water and filtered in a Buchner funnel. This step was repeated until the resulting filtrate was clear and at neutral pH. The bleached processed fiber was dried in an air-circulated oven at a temperature of 35-60° C. and then ground to 100-mesh size (e.g., 150-250 micron) using a Retsch mill.

### B. Papermaking: Laboratory Investigation of Effect of Additive (EFA-C) on Paper Properties

Papermaking Furnish Preparation: Hardwood and softwood bleached Kraft commercially available market pulp was received from Georgia Pacific. A 50% hardwood and 50% softwood blend was slurred with distilled water to 1.2% by weight consistency in a 5-gallon container. 0.5% by weight of EFA-C (Enhanced Fiber Additive made from Corn Fiber) was added to the 1.2% consistency hardwood/softwood papermaking slurry.

Refining: Tappi Method T-200 describes the procedure used for laboratory beating of pulp using a valley beater. The hardwood/softwood papermaking pulp furnish containing the EFA-C was refined using a valley beater. The furnish was refined to 450 mL CSF (Canadian Standard Freeness). The freeness of the pulp was determined using the TAPPI test method T-227. Once 450 mL CSF was obtained, the finish was diluted to 0.3% consistency with distilled water and gently stirred with a Lightning mixer to keep the fibers in the papermaking furnish suspended.

Making Handsheets: Paper was made using the following handsheet procedure according to TAPPI Test Method T-205. Basis weights of 1.2 gram handsheets (40 lb sheet or 40 lb/3300 ft² or 60 g/m²) and 1.8 gram handsheets (60 lb sheet or 60 lb/3300 ft² or 90 g/m²) were for comparison. In some instances, 20 lb/ton of a cationic dent corn starch (Charge +110 from Cargill) was added to the handsheet mold to aid in drainage and retention.

Handsheet Testing: The paper handsheets were submitted to Integrated Paper Services (IPS, Appleton, Wis.). The paper handsheets were conditioned and tested in accordance to TAPPI test method T-220 Physical Testing of Pulp handsheets. Instruments used: Caliper—Emveco Electronic Microguage 200A; Burst—Mullen Burst Test Model “C”; Tear—Elmendorf Tear Tester; Tensile—SinTech.

Results: Table 1 represents the paper properties from the handsheet evaluation with and without the EFA-C.

TABLE 1
Handsheet Paper Test Results

| | | | | | | Burst Index | | Tear Index | | Tensile Index | |
|---|---|---|---|---|---|---|---|---|---|---|---|
| | | Caliper (mils) | | Basis Wt (g/m²) | | (kPA m²/g) | | (mN m²/g) | | (N-M/g) | |
| | Handsheet | No | 20 lb/t | No | 20 lb/t | No | 20 lb/t | No | 20 lb/t | No | 20 lb/t |
| | (g) | Starch | CH + 110 | Starch | CH + 110 | Starch | CH + 110 | Starch | CH + 110 | Starch | CH + 110 |
| Control | 40 lb | 20.25 | 21.00 | 63.22 | 64.72 | 3.38 | 3.70 | 12.81 | 13.00 | 46.96 | 52.33 |
| Control | 60 lb | 29.39 | 29.46 | 97.75 | 95.21 | 3.76 | 4.20 | 14.64 | 13.25 | 51.49 | 55.34 |
| EFA-C | 40 lb | 20.00 | 21.41 | 66.01 | 69.42 | 3.80 | 4.33 | 11.60 | 11.21 | 51.54 | 58.59 |
| EFA-C | 60 lb | 28.91 | 29.59 | 101.65 | 102.35 | 4.05 | 4.67 | 13.58 | 12.39 | 57.78 | 59.14 |

The study demonstrated the enhanced burst strength with the addition of 20 lb/ton of cationic starch and also as a result of EFA-C presence. Note the 60 lb sheet without the EFA-C (control) has equivalent burst strength to the 40 lb sheet with 0.5% EFA-C.

The study also demonstrates the enhanced tensile strength with the addition of 20 lbs/ton of cationic starch and also as a result of EFA-C presence. Note the 60 lb sheet without EFA-C (control) has at least equivalent tensile strength to the 40 lb sheet with 0.5% EFA-C.

Conclusion: A 40 lb sheet made in the laboratory with 0.5% EFA-C retains equivalent burst and tensile strengths as a 60 lb sheet without EFA-C. A catalytic amount of EFA-C (0.5 %) replaced 33% of the Kraft wood fiber in a standard 60 lb sheet without sacrificing burst and tensile strengths. The addition of 20 lb/ton of cationic starch also elevated burst and tensile properties.

### C. Pilot Investigation Comparing Agricultural Additives (EFA and Cationic Starch) With Respect to Paper Properties

A pilot paper machine trial was performed at Western Michigan University in the Paper Science & Engineering Department. The objective of the trial was to determine if the paper strength enhancement properties of the EFA-C would be changed by the addition of cationic starch.

Papermaking Furnish Preparation: Hardwood and softwood bleached Kraft commercially available market pulp was supplied by Western Michigan University. Two different batches of a 60% hardwood and 40% softwood furnish were prepared for the study. One batch contained no EFA-C and was labeled “Control.” The other batch contained 2.0% EFA-C and was labeled “EFA-C” batch. Each batch was prepared as follows: A 5% by weight consistency of 60% hardwood and 40% softwood was blended and mixed together in the Hollander Beater. Tap water was used to achieve the 5% consistency. Once the pulp was blended and re-hydrated with water, the pulp slurry was transferred to the Back Chest and diluted to 1.5% with tap water. The pH of the slurry was adjusted to a pH of 7.5 with H₂SO₄. From the Back Chest, the pulp slurry was sent through a single disc Jordon refiner until a freeness of 450 mL CSF was achieved. The freeness was determined by TAPPI Test Method T-227. A load weight of 40 lbs and flow rate of 60 gpm were the operation parameters assigned to the Jordon refiner. The refining time of each batch was kept constant (12 minutes). The EFA-C material was added to the Back Chest prior to refining at a dosing level of 2.0% by weight of the EFA-C. Once refining was completed, the pulp slurry was transferred to the Machine Chest and diluted to 0.5% consistency.

Making Paper: Two different basis weight grades of paper were targeted, a 36 lb/3300 ft² and a 73 lb/3300 ft². Basis weights were achieved by controlling the machine speed. When called for during the experiment, 10 lb/ton of cationic starch (Charge +110) was added at the Stuffbox. The 0.5% slurry was transferred from the Machine Chest to the Headbox. From the Headbox the slurry was transferred to the Fourdrinier as described previously. The Size Press and Second Dryer sections were by-passed as before. The final stage of the web passed through the Calender Stack and onto to the Reel.

Paper Testing: All the paper testing was performed by Western Michigan University-Paper Science & Engineering. Table 2 represents the references to the TAPPI Test Procedures and number of replications performed on each test.

TABLE 2
TAPPI Test Methods

| Test Identification | TAPPI Method | Replications |
|---|---|---|
| Basis Weight | T-410 om-93 | 5 |
| Ash Content | T-413-om-93 | 3 |
| Bulk | T-220 sp-96 14.3.2 | 10 |
| Gurley Porosity | T-460 om-96 | 10 |
| Caliper | T-411 om-89 | 10 |
| Tensile Strength | T-494 om-88 | 10e/10 CD |
| Opacity | T-425 om-91 | 5 |
| Tearing Force | T-414 om-88 | 5 |
| Scott Bond | T-541 om-89 | 5 |
| Burst Strength | T-403 om-91 | 10 wire side/10 felt side |
| Gurley Stiffness | T-543 om-94 | 5 MD/5 CD |
| Folding Endurance | T-511 om-96 | 10 MD/10 CD |
| Sheffield Roughness | T-538 om-96 | 10 wire side/10 felt side |

Results: The results of the paper testing are shown in Table 3.

TABLE 3
Western Michigan University Pilot Paper Machine Trial

| | | | | | | | Tensile | |
|---|---|---|---|---|---|---|---|---|
| | | Cationic | Actual | | Gurley | | Index | |
| Grade | EFA-C | Starch | Basis Weight | Bulk | Porosity | Caliper | (N m/g) | |
| (lb/3300 ft2) | (%) | (lb/ton) | (lb/3300 ft²) | (cm³/g) | (sec/100 mL) | (mils) | MD | CD |
| 36 | 0 | 0 | 37.61 | 2.82 | 3.28 | 3.47 | 29 | 13 |
| 36 | 0 | 10 | 36.82 | 2.79 | 3.04 | 3.36 | 54 | 32 |
| 36 | 2 | 0 | 37.46 | 2.63 | 3.54 | 3.23 | 29 | 12 |
| 36 | 2 | 10 | 37.27 | 2.69 | 4.10 | 3.29 | 37 | 15 |
| 73 | 0 | 0 | 69.63 | 2.78 | 6.02 | 6.34 | 58 | 31 |
| 73 | 0 | 10 | 73.52 | 2.84 | 6.33 | 6.83 | 76 | 37 |
| 73 | 2 | 0 | 73.46 | 2.62 | 7.72 | 6.31 | 60 | 29 |
| 73 | 2 | 10 | 72.41 | 2.71 | 8.52 | 6.18 | 78 | 37 |
| | | Cationic | | Tearing Force | | Scott | Burst Index | |
| Grade | EFA-C | Starch | Opacity | (gf) | | Bond | (KPa g/m²) | |
| (lb/3300 ft2) | (%) | (lb/ton) | (%) | MD | CD | (ft lb/1000 in²) | Wire | Felt |
| 36 | 0 | 0 | 77 | 67 | 67 | 106 | 1.17 | 0.97 |
| 36 | 0 | 10 | 76 | 65 | 81 | 143 | 2.87 | 2.99 |
| 36 | 2 | 0 | 74 | 54 | 68 | 126 | 1.03 | 1.00 |
| 36 | 2 | 10 | 74 | 61 | 69 | 173 | 1.37 | 1.35 |
| 73 | 0 | 0 | 89 | 143 | 132 | 109 | 2.37 | 2.39 |
| 73 | 0 | 10 | 88 | 157 | 167 | 157 | 3.01 | 3.29 |
| 73 | 2 | 0 | 86 | 126 | 128 | 126 | 2.45 | 2.30 |
| 73 | 2 | 10 | 85 | 136 | 143 | 160 | 3.24 | 3.10 |

| | | | | | | | Sheffield | |
|---|---|---|---|---|---|---|---|---|
| | | Cationic | Gurley Stiffness | | Folding Endurance | | Roughness | |
| Grade | EFA-C | Starch | (gurley units) | | (log10 MIT) | | (mL/min) | |
| (lb/3300 ft2) | (%) | (lb/ton) | MD | CD | MD | CD | Wire | Felt |
| 36 | 0 | 0 | 225 | 71 | 1.22 | 0.58 | 202 | 230 |
| 36 | 0 | 10 | 204 | 98 | 1.62 | 0.90 | 192 | 230 |
| 36 | 2 | 0 | 215 | 68 | 1.12 | 0.54 | 177 | 207 |
| 36 | 2 | 10 | 185 | 38 | 1.53 | 0.88 | 180 | 213 |
| 73 | 0 | 0 | 390 | 164 | 1.73 | 1.11 | 232 | 284 |
| 73 | 0 | 10 | 420 | 194 | 2.17 | 1.48 | 237 | 287 |
| 73 | 2 | 0 | 330 | 158 | 1.55 | 0.90 | 222 | 279 |
| 73 | 2 | 10 | 376 | 165 | 1.99 | 1.29 | 223 | 264 |

Conclusions: The addition of 2.0% EFA-C increased the internal bond strength of paper as measured by the Scott Bond TAPPI test method. When 2.0% EFA-C was incorporated into the paper, the sheet became less porous. The Bulk density of the paper increased with the addition of 2.0% EFA-C. Incorporation of a cationic starch with the 2.0% EFA-C into the paper enhances the properties described above. The pilot paper machine study also indicated that there is a synergistic effect of using the EFA in conjunction with a cationic starch with respect to machine runnability parameters of drainage and retention.

### D. Analysis of Agricultural Additive Distribution of (EFA-C) in Paper Products

Papermaking: Analysis of EFA-C in Paper Products

The objective of the study was to determine whether a test method could be developed which identified the EFA technology in a paper product using either a microscopic and/or spectroscopic technique. Paper was made with different concentrations of EFA-C on the pilot paper machine at Western Michigan University Paper Science & Engineering Department.

Papermaking Furnish Preparation: Hardwood and softwood bleached Kraft commercially available market pulp was supplied by Western Michigan University. Different batches of a 60% hardwood and 40% softwood were prepared for the study. Each batch contained one of the following levels of EFA-C: 0%, 0.5%, 1.0%, and 2.0%. Each batch was prepared as follows: A 5% by weight consistency of 60% hardwood and 40% softwood was blended and mixed together in the Hollander Beater. Tap water was used to achieve the 5% consistency. Once the pulp was blended and re-hydrated with water, the pulp slurry was transferred to the Back Chest and diluted to 1.5% with tap water. The pH of the slurry was adjusted to a pH of 7.5 with H₂SO₄. From the Back Chest, all of the pulp slurry was sent through a single disc Jordon refiner three times. A freeness of 480 mL CSF (TAPPI Test Method T-227) was measured. A load weight of 20 lbs and flow rate of 60 gpm were the operation parameters of the Jordon refiner. The refining time of each batch was kept constant. The furnish was drawn from the Back Chest through the single disc Jordon refiner and onto the Machine Chest. Once the Back Chest was drawn empty, the Jordon refiner was turned off. The batch was then transferred from the Machine Chest back to the Back Chest. This process was repeated three times for each batch containing different levels of EFA-C. Once refining was completed, the pulp slurry was transferred to the Machine Chest and diluted to 0.5% consistency.

Making Paper: Three different basis weight grades of paper were targeted at 20, 40, and 60 lb/3300 ft². Basis weights were achieved by controlling the machine speed. For runability purposes, 10 lb/ton of cationic starch (Charge +110) was added at the Stuffbox. The 0.5% slurry was transferred from the Machine Chest to the Headbox. From the Headbox the slurry was transferred to the Fourdrinier as described previously. The Size Press and Second Dryer sections were by-passed as before. The final stage of the web passed through the Calender Stack and onto to the Reel.

SEM Studies

The paper samples were subjected to standard Scanning Electron Microscopy examination in order to determine if any structural changes were occurring as a result of the usage of the EFA-C in the paper making process. FIG. 1 shows an SEM image at 100×, and FIG. 3 an SEM image at 800× of a 40 lb sheet made in the manner described above, with 0% added EFA-C. In FIG. 3 microfibrils that connect the fibers, as well as the large void spaces, are observable in the paper surface. The presence of micro-fibrils is known to increase the strength of the paper sheet (T. E. Conners and S. Banerjee in Surface Analysis of Paper, CRC Press, 1995). FIG. 2 shows an SEM image at 100× and FIG. 4 at 800× of a 40 lb sheet made with 1% EFA added before the refining step. Note the increase in micro-fibril production (FIG. 4) in this example. Also note that the void spaces are now reduced, indicating a better formation of the paper sheet.

In total, a 23% increase in micro-fibril production was noted in the above paper sheets. Calculations were performed on 20 SEM field images of paper without EFA addition and 20 SEM field images of paper with 1% EFA-C addition. Paper without EFA averaged 13 micro-fibrils per micrograph field and paper with 1% EFA-C averaged 16.5 micro-fibrils per micrograph field, thus an increase of 23% over non-EFA paper.

Fourier Transform Infrared Spectral Analysis

Infrared spectral analyses of paper handsheets were performed. Fourier transform infrared reflectance spectra of 40-lb sheet with no EFA-C added and of 40-lb sheet with 1% EFA-C were scanned. FIG. 7 shows the results of the test. The spectrum at A in FIG. 7 spectrum is from paper with no EFA-C added; and the spectrum at B in FIG. 7 is paper with 1% EFA-C additive. Spectrum C, FIG. 7, shows the residual after spectra subtraction using simple 1:1 ratio factor; i.e., shows the differences between A and B. The region of most difference in the two spectra is circled in the figure; in general, in the region between 1100 cm⁻¹ and 1300 cm⁻¹ centered on peaks at about 1200 cm⁻¹.

Chemical Imaging

FIG. 5, shows a chemical morphology NIR-based image contrast plot of non-EFA paper, and FIG. 6 shows such a plot of EFA paper.

The NIR data was collected over the range of 1000 to 1700 nm, every 5 nm. The samples were 60 microns thick.

The images were generated by using a principal component analysis (PCA) as characterized above. This type of technique enhanced the chemical differences found in the “principal components” of the variations in the material examined. The images shown in FIGS. 5 and 6 are of the third principal component of the paper image. In chemical imaging, the contrasts generated in the image are from chemical, rather than physical, differences. The measurements used, and imaging analysis, were performed by ChemIcon, Inc. at Pittsburgh, Pa., using that company's facilities and software, under the supervision of Cargill, Inc., the assignee of the present application.

Note that the non-EFA material (FIG. 5) shows very little contrasting chemical morphology. This implies a fairly homogenous chemical makeup. However, the image of the EFA added paper (FIG. 6) shows marked contrasts. That is, there are localized chemical differences across this image. In fact, on close examination of the EFA image, one can see that the chemical changes generated by the presence of the EFA material are localized or ordered to follow (or to align and define) individual paper (in this case pulp or cellulose) fiber strands. That is, the EFA is located such that it coats, or at least partially coats, various paper fibers (i.e., cellulose or pulp fibers in this instance). Since the EFA material has a significant holocellulose character, it readily interacts with the wood (cellulose) fibers. Because of its hemicellulose character, the EFA acts as “glue” in paper manufacturing. Thus, it can be concluded that the EFA additive effectively coats (or partially coats) each paper (holocellulose) fiber with a thin film of hemicellulosic “glue” and in this manner adds to the overall strength of the paper.

In order to ensure that the PCA 3 image contrast is from EFA, a small piece of ground EFA material was placed upon the paper and imaged in principal component space.

It is noted that when the experiment was performed, and the differences were plotted by the researchers, the chemical differences were plotted in color, to enhance contrasts in the images generated. Non-color images are provided in the current figures; however the contrasts are still apparent.

It is also noted that when the experiment was first performed, plots were also made of loadings corresponding to PC1, PC2, PC4 and PC5; however, these showed relatively little differences between the samples.

Quantitative Analysis

Once it was observed that EFA could be detected spectrally, and even imaged spectrally, it was concluded that a quantitative spectral model could be developed. Such a model would enable one to determine not only if EFA material were present in the paper, but to determine how much EFA is present.

A calibration data set was put together with 0% and 1% EFA additive to demonstrate that a quantitative spectra model could be developed. By recording the reflectance near infrared spectrum of each paper sample, a spectra correlation plot was developed. This data is presented in U.S. Ser. No. 09/689,994, incorporated by reference.

Conclusions

The following conclusions can be made about the paper application of EFA, based upon the above experiments and analysis:

1. The paper enhancements of EFA occur with very little differences occurring visually (SEM at 100× or 800×) in bulk structure between paper with and without EFA.

2. Differences in infrared spectra characteristics are observable, showing that there are chemical differences between EFA and cellulose.

3. Differences in FTIR spectra are real and overtones of these bands are present in NIR correlation analysis.

4. NIR imaging graphically shows EFA localized chemical differences from EFA addition, in the form of “coating” of the paper (cellulose) fibers. This effect contributes to the strength building characteristics of EFA.

5. The spectral differences are large enough to develop an analytical method for EFA in paper using NIR.

### EXAMPLE 2

### Non-Destructive Determination of Cocoa Butter Distribution in Processed Cocoa Powders

The purpose of this study was to compare a new cocoa sample to commercial cocoa samples. A series of cocoa powders with known fat content were used to generate a calibration used to determine the fat content and distribution in a new cocoa sample.

Cocoa is processed from the cocoa or cacao bean into various forms using different chemical and physical treatments. After fermentation and drying, the fat content of the raw bean in the nib is ˜60%, a majority of which is cocoa butter, according to the International Cocoa Organization. Cocoa butter is composed primarily of monounsaturated glycerides (see table 4) and is extracted from the bean via mechanical press or solvent extraction. After butter extraction, the beans are ground and further processed by alkalization, which alters the color and the flavor of the finished cocoa.

TABLE 4
Cocoa butter composition in cacao beans prior to processing.

| Glycerides | Percentage |
|---|---|
| Trisaturated | 2.5 to 3.0 |
| Triunsaturated (triolein) | 1.0 |
| Diunsaturated | |
| Stearo-diolein | 6 to 12 |
| Palmito-diolein | 7 to 8 |
| Monounsaturated | |
| Oleo-distearin | 18 to 22 |
| Oleo-palmitostearin | 52 to 57 |
| Oleo-dipalmitin | 4 to 6 |

Five ground cocoa samples were evaluated by Near Infrared Hyperspectral Imaging with the goal of determining differences in cocoa butter content and distribution. These samples are designated as 10/12 and 22/24 Amber and/or Garnet. The numerical designator is the fat range of the cocoa (10% to 12% or 22% to 24%) and the amber or garnet designation refers to the processing of each cocoa; garnet is alkalized; and amber is unalkalized. The fifth sample in the study was a new cocoa sample.

A near infrared (NIR) microscope was used for the acquisition of hyperspectral diffuse reflectance images in the range of 1100 to 1650 nM using a 20× NIR corrected objective. The system was equipped with a high intensity tungsten-halogen illuminator and a visible Bright field camera for acquisition of field of view (240×320 micrometer field of view) RGB (red, green, and blue) images. The reflected light intensity from diffusely reflecting dark powders was low compared with a standard reference material, and therefore the high intensity illuminator was needed to achieve the best signal to noise characteristics in the spectroscopic data. There was some concern that the higher intensity light would melt the cocoa powder in the regions of high fat, as butter melting occurred when the acquisition of the cocoa butter and alkalized liquor images was attempted. However, the high intensity tungsten lamp did not significantly alter the cocoa powders upon exposure.

Each cocoa powder sample was spread onto a microscope slide and flattened with a coverslip in order to provide a reasonably uniform surface for imaging. The coverslip was removed prior to image acquisition and reference images (Spectralon™ NIR reference material) were obtained in sequence after each sample scan. The data were processed into absorbance images (-log₁₀(I/Iₒ)), where higher values correspond to higher absorption of incident light by the sample. Qualitative or quantitative analysis may be performed on the resulting image cubes in “image space”, where each frame contains the pixel-by-pixel absorbance values for the sample at the specific frame wavelength. Likewise, one may choose to plot an average spectrum for a given region of interest, where the x-y plot (spectrum) is representative of the chemical information contained in the image.

In order to locate and quantitate the cocoa butter in the cocoa samples, a focus was made on the 1150 nm to 1225 nm region where glycerides absorb NIR light due to the second overtone of the aliphatic stretching mode. Regions of higher fat content will yield stronger absorptions in this spectral range, and one therefore may use this region in an attempt to quantify the distribution of cocoa butter in the cocoa powder samples.

The first task upon data collection was to analyze the bulk spectra obtained for the entire field of view in order to determine if any gross differences existed that could be related to fat content. NMR spectra were obtained by averaging spectra from each pixel in the entire field of view for each image cube and may be regarded as analogous to bulk NIR diffuse reflectance spectra. The spectra (not shown) exhibit a key attribute that is different from traditional bulk NIR spectra, i.e., the rising baseline toward the blue end (lower wavelengths) of the spectrum.

Bulk NIR spectra of natural products tend to have a baseline that increases with wavelength due to scattering effects in the matrix. A rise in the baseline as one moves to lower wavelengths is indicative of scattering effects due to the presence of particles or features which are significantly smaller than the interrogation wavelength (Rayleigh scattering). One reason for this type of scattering artifact is that the illumination and collection plane (sampling plane) may minimize the effects of diffuse reflectance scattering due to the lack of efficiency in collecting out-of-plane diffusely scattered light. This in turn would emphasize the scattering due to in plane particles that have either particle sizes or surface morphologies with feature sizes considerably smaller than the interrogation wavelength. However, the spectra contained features above the scattering baseline at 1200 nm that are due to true optical absorption of aliphatic compounds in the fields of view.

The first step in further data processing was the removal of baseline offsets that are not the result of chemical structural differences. A linear baseline subtraction was applied to the spectral axis of each image centered around, but not including, the aliphatic absorption around 1200 nm. The baseline corrected average spectra clearly differentiated samples based on cocoa butter, or fat, content. The absorbance values at 1205 nm were tabulated for each baseline corrected image and these were used to generate a linear least squares model relating the fat content, as indicated by the sample identification, to absorbance. This relationship was used to generate a pseudocolor image where the intensity of the color (or gray scale) is directly proportional to fat content. In this disclosure the fat distribution is exhibited as gray scale images separate from the Bright field images.

Specifically, for each sample, a 16-bit gray scale image was extracted at 1205 nm from baseline corrected data cubes. This image was then corrected to remove cosmic rays and then processed at each pixel using the following algorithm, obtained from the regression of % fat and average absorbance at 1205 nm:

% fat=2770.5*(A_{1205 nm})−0.2108

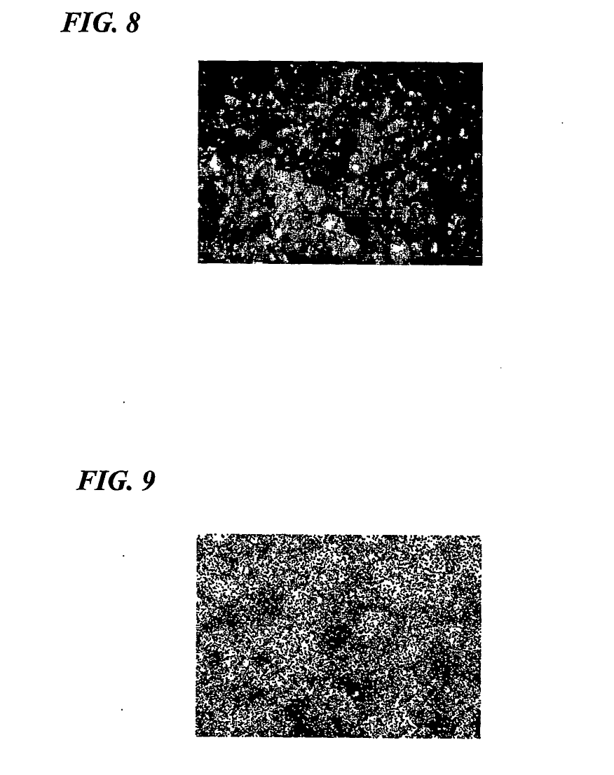

The preceding relationship converts each pixel absorbance value at 1205 nm to the percent fat in the cocoa represented at that pixel position, in turn generating an estimate of the spatial distribution of cocoa butter in the image plane, assuming that a majority of the aliphatic absorption at 1205 nm is due to the presence of cocoa butter glycerides. FIGS. 8 through 11 display both Bright field images and corresponding fat distribution images (chemical morphology NIR-based image contrast plots) collected with the NIR microscope; i.e., chemical morphology NIR-based image contrast plots. The threshold for the binary fat images is set at 20% fat, i.e., areas of the image that are gray represent regions of the image that contain greater than 20% fat. This threshold yields a useful distribution image while not obscuring the Bright field image when viewed in overlay.

Referring to FIGS. 8 through 11:

1. FIG. 8 is a Bright field image (20×) of 22/24 garnet cocoa (320×240 micrometer field of view);

2. FIG. 9 is a fat distribution image (chemical morphology NIR-based image contrast plot) for 22/24 garnet cocoa showing regions in the field of view having greater than a selected fat content (in this case, above a 20% fat content) (higher fat regions being represented by dark pixels);

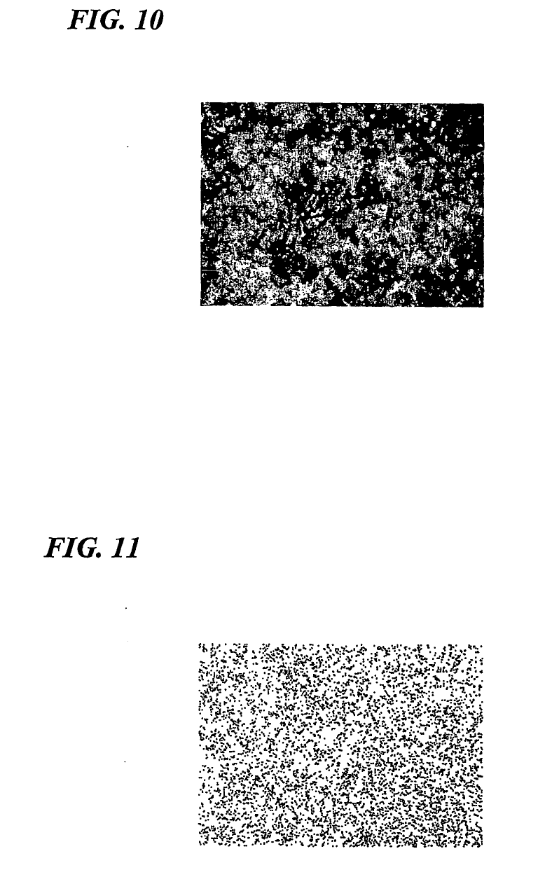

3. FIG. 10 is a Bright field image (20×) of the new cocoa sample (320×240 micrometer field of view); and

4. FIG. 11 is a fat distribution image (chemical morphology NIR-based image contrast plot) for the new cocoa sample showing regions in the field of view having greater than 20% fat content (higher fat regions being represented by dark pixels).

In FIGS. 8 and 9 the 22/24 (high fat) cocoa samples display a greater amount of fat distributed in the images as well as larger regions of higher fat content. (FIG. 9 can be placed over FIG. 8 as an overlay, since both are from the same sample region.) In addition, the higher fat density regions are located primarily in the interstitial sites between larger cocoa particles. The higher fat content cocoa powders are processed for either a shorter time or under lower press pressures. These less extreme processing steps would move of the fat out of the nib and into the interface regions but not out of the cocoa mass. As the pressure is increased, butter is extruded through the interstitial regions and away from the cocoa mass. Therefore, it is intuitive that the fat in the higher fat cocoa would occupy the interface regions between individual nib particles.

The new cocoa sample appeared to be similar to the 10/12 amber cocoa (not shown) in physical appearance, spectroscopic properties, and fat distribution. FIGS. 10 and 11 show the Bright field image (FIG. 10) and the chemical morphology NIR-based image contrast plot (FIG. 11), for >20% fat distribution image of the new sample. (FIG. 11 can be placed on FIG. 10 as an overlay, since both are from the same sample region.) The new cocoa sample had significantly less cocoa butter distributed throughout the field of view, as expected by comparison to the 22/24 sample. It also appears to have a relatively even distribution of cocoa butter, with no large particles of high fat visible in the field of view. Therefore, differences in cocoa butter distribution between the 22/24 cocoa and the new cocoa sample indicate the new sample contains significantly less cocoa butter than the 22/24 sample. (indeed, the new sample compared favorably with the 10/12 cocoa butter, although the comparison images are not given here, for brevity.)

### EXAMPLE 3

### Chicken Skin Emulsion Imaging by Near Infared Hyperspectral Imaging

Several samples of chicken skin emulsions were analyzed using Near Infrared hyperspectral imaging with the goal of determining fat and water distribution differences between each emulsion type. The emulsions were prepared using proprietary methods, after which each sample was shipped to the analysis facility after preparation. Therefore, the preparation of the emulsions will not be discussed in this report.

Chicken Skin Emulsion (CSE) is an emulsion made from chicken skin, soy protein isolate, water and salt. The primary difference between the two products analyzed in this study is in the manufacturing process used where the two products are representative of the two predominant methods for manufacture of poultry emulsions. The Type 2 process appears to result in relatively more stable, and therefore more desirable, emulsions relative to the Type 1 process. Thus, it was hypothesized that the Type 2 emulsions would exhibit smaller fat (lipid) domain sizes relative to the less stable Type 1 emulsion. In order to investigate this, two imaging techniques were utilized: Bright field microscopy and near infrared (NIR) imaging. The results are presented here in 16-bit gray scale image format, however, the preferred method for data visualization will typically be an indexed false-color image.

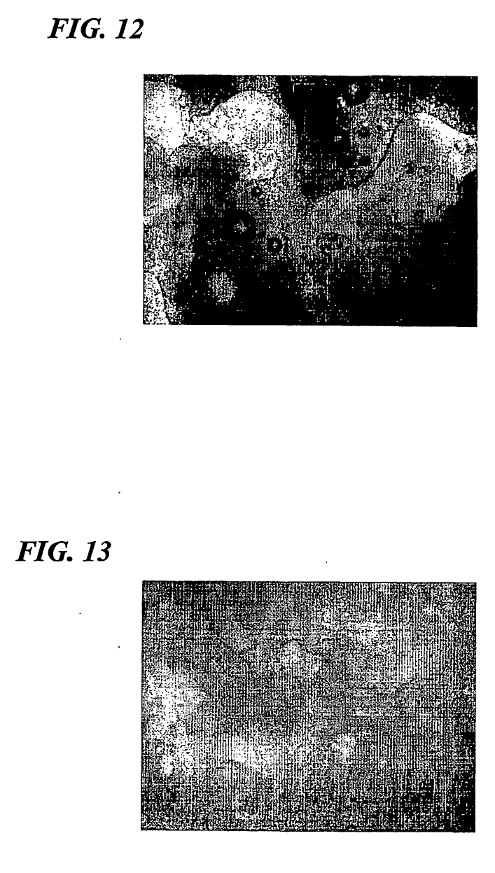

Samples for microscopic imaging were prepared by placing a small amount of each chicken skin emulsion on a glass microscope slide and then creating a thin emulsion layer by pressing the sample with a 0.17 mm glass cover slip. Bright field images were acquired using a standard Bright field microscope in transmission mode under Koehler illumination. FIG. 12 shows a transmission Bright field image of the Type 1 CSE product. Similarly, FIG. 13 displays the corresponding Bright field transmission image of the Type 2 CSE product. Each CSE sample contained features that appear to be irregularly shaped vesicles, with both relatively dark and light vesicles present. An assessment of sample heterogeneity may be made from the Bright field images, and this assessment may be made without regard for the chemistry of the vesicles, i.e., without regard for whether they are pure water and/or fat components or simply air pockets in the CSE materials. Based on simple image inspection, the Type 1 sample contains larger vesicles, and is therefore qualitatively more heterogeneous than the Type 2. (The dark-ringed spherical objects in the Type 1 image are due to the refractive index effects associated with trapped air bubbles within the sample.)

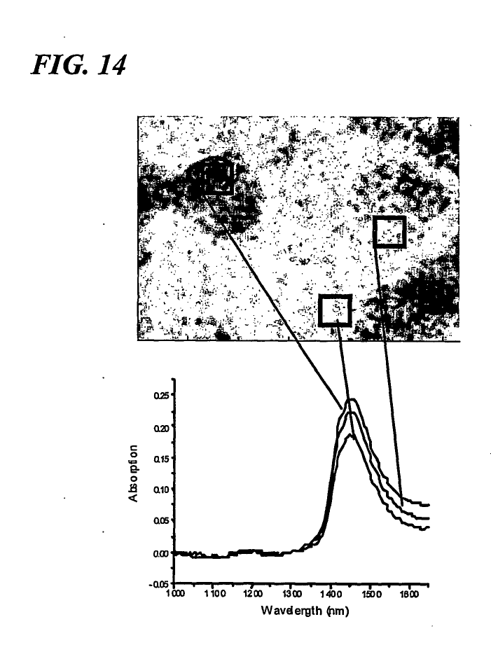

FIGS. 14 and 15 are the gray scale images (chemical morphology NIR-based image contrast plots) representing the absorption of incident light at 1440 nm. In these Figs., spectra are included to show correlation. FIG. 14 is of the Type 1 CSE. FIG. 15 is of the Type 2 CSE.

The absorption at this wavelength (1440 nm) is due to the first vibrational overtone of —OH and is attributed primarily to water. In these images, the absorbance is represented by the gray level, where darker regions correspond to higher absorbance values. (Absorbance values were calculated using raw, reference and background images using standard spectroscopic calculation methods). Spectra were baseline corrected at 1000 nm and 1300 nm and fitted with a single order polynomial. Again, NIR absorbance spectra are shown below their respected images.

Several observations can be made by comparing the Bright field and NIR images. First, for each image, the entire field of view contains water with variable concentration across each field of view. Second, vesicles that appear clear in the Bright fields images are dark gray in the NIR 1440 nm absorbance images. This implies that the visibly clear vesicles contain a higher concentration of water than the dark vesicles due to the lower transmission of light at 1440 nm through the visibly clear vesicles.

In summary, the Bright field and chemical imaging studies of two different types of chicken skin emulsions, Type 1 and Type 2, show that they are significantly different. Physically, Type 1 CSE is smooth and paste-like while Type 2 CSE is firm and rubbery. Moreover, under 20× Bright field microscopic evaluation, Type 1 is significantly more heterogeneous than Type 2 CSE. The emulsions are comprised of two vesicles: ‘clear’ and ‘dark’, as seen in the visible Bright field images. As can be readily observed by microscopic NIR chemical imaging, clear vesicles in each visible field of view contain higher water concentration relative to the dark vesicles. Based on this spectroscopic evidence, the clear vesicles are aqueous in nature while the dark vesicles are more organic in nature.

This study shows the utility of near infrared hyperspectral imaging for determining the microscopic distribution of water and fat species in an emulsion. In addition, comparisons are made between samples having different functional properties, allowing correlations to be drawn between the chemical images and functionality of the materials in a product or formulation.

### EXAMPLE 4

### Agglomerated Cocoa Power Study

Several defatted cocoa powder samples were analyzed by Near Infrared Hyperspectral Imaging (NIHI). The goal of the research was to find differences in the samples that correlate with the dispersability and wetting characteristics of the cocoa powders in milk or water. The ability of NIHI to determine differences in concentration and distribution of sucrose in the cocoa powders is demonstrated. The sucrose (a sugar, specifically a disaccharide) is used as a sweetener and a dispersion agent to aid in the wettability of the cocoa powders.

Several agglomerated and unagglomerated defatted cocoa powder samples were received for analysis. The cocoas were produced using different processing techniques for fat removal and agglomeration. The agglomeration process involved either steam agglomeration or a cold agglomeration using sucrose solution and the actual agglomeration method was unknown at the time of analysis. Two cocoas will be discussed in this report: a reference sample, comprising a commercial defatted cocoa with added sucrose; and, a pilot sample, comprising a pilot process defatted cocoa with added sucrose.

Near Infrared Hyperspectral Imaging Results

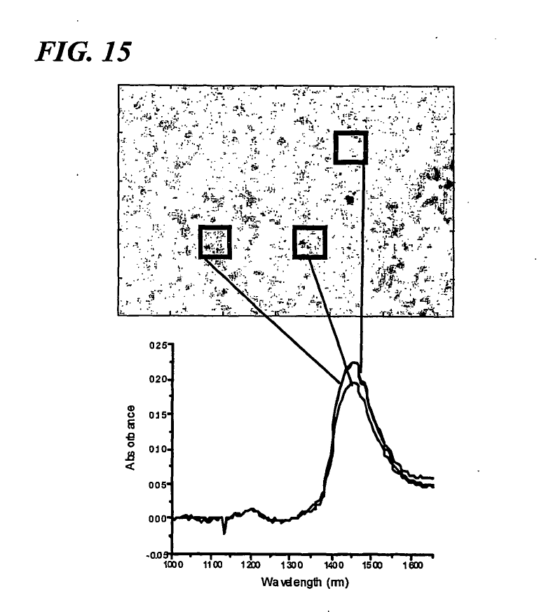

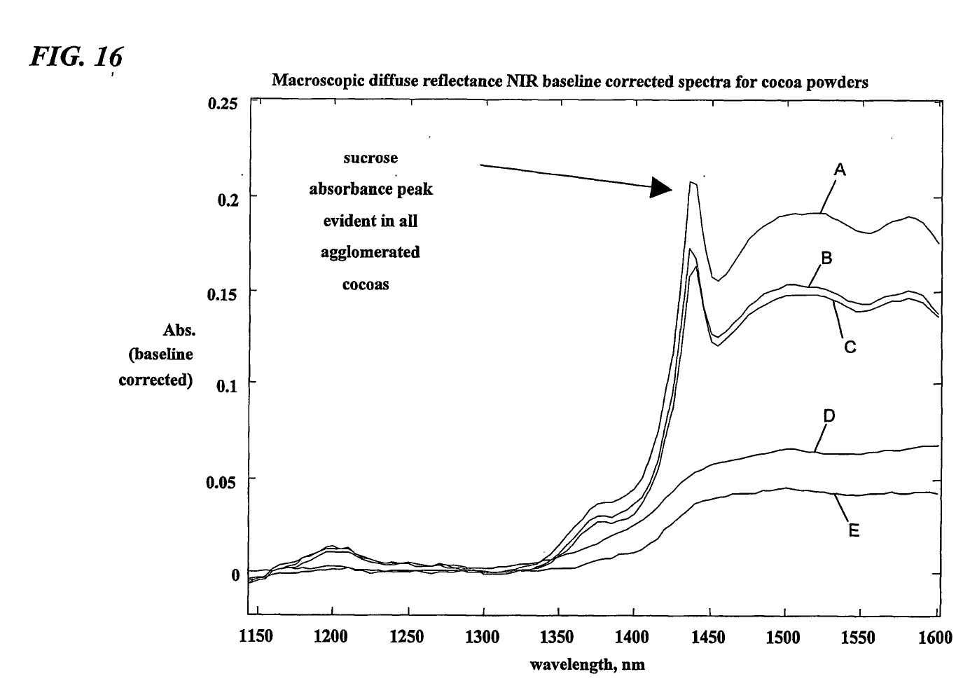

NIHI images were collected for all five cocoa samples using macroscopic (5× objective, 3.375×2.475 mm field of view, 10×10 micron pixel resolution) and microscopic (20× microscope objective, 320 by 240 micron field of view, 1×1 micron pixel resolution) systems. The samples were placed on microscope slides and flattened using a coverslip in order to provide a uniform focus across the field of view. Hyperspectral images were acquired in the range of 1000 to 1600 nm at best focus for each sample on each imaging system. Baseline corrections using non-absorbing spectral regions were applied to the data in order to remove the effects of scattering. Average field of view spectra are presented in FIG. 16 for a macroscopic field of view. The grayscale spectra are presented for all 5 cocoas analyzed in the study. The spectra corresponding to cocoa samples with added sucrose form the basis for further mapping sucrose as there are clear differences in average sucrose concentrations between samples. Microscopic field of view average spectra are similar but of lower magnitude, and, although discussed briefly, are not presented in this example. In FIG. 16, the spectra lines A, B, C, D, and E correspond to the five examples; with three samples (A, B, and C) showing the sucrose, and two samples (D and E) not having the sucrose peak. Samples A, B, and C were agglomerated samples. Samples D and E were unagglomerated cocoa powders for reference.

The sharp peak at 1435 for all agglomerated cocoas indicates that the cocoas were processed into agglomerated products by sucrose addition. This sharp peak is a signature of the beta-D-glucose moiety present in the disaccharide sucrose. The low intensity feature at 1200 nm is due to 2^{nd} overtone of carbon-hydrogen bond vibrations. These are normally attributed to fat present, but in this case, they arise from the added sucrose. Notice that the traces for the defatted cocoa (unagglomerated) show very little intensity at 1200 nm, indicating again that the intensity at 1200 is almost entirely due to the sucrose addition. This is evidence that all of the cocoas were indeed devoid of measurable fat content.

The differences between the relative microscopic and macroscopic sucrose peak magnitudes are related to the difference in the fields of view: the microscopic field images fewer particles, and therefore the chances of finding an average field of view representative of the entire sample are lower. In contrast, the macroscopic image field provides less detail but is more representative of the bulk properties of the sample. Combining the two techniques allows generalizations to be made independent of field of view. A result of this analysis is the determination that the agglomerated reference cocoa contains a higher concentration of sucrose than the agglomerated pilot process cocoas. The presence of sucrose in the system enhances wettability as well as dispersability. Therefore, a difference in dispersability between cocoas is most likely related to the amount of sucrose infused into the cocoa powder system.

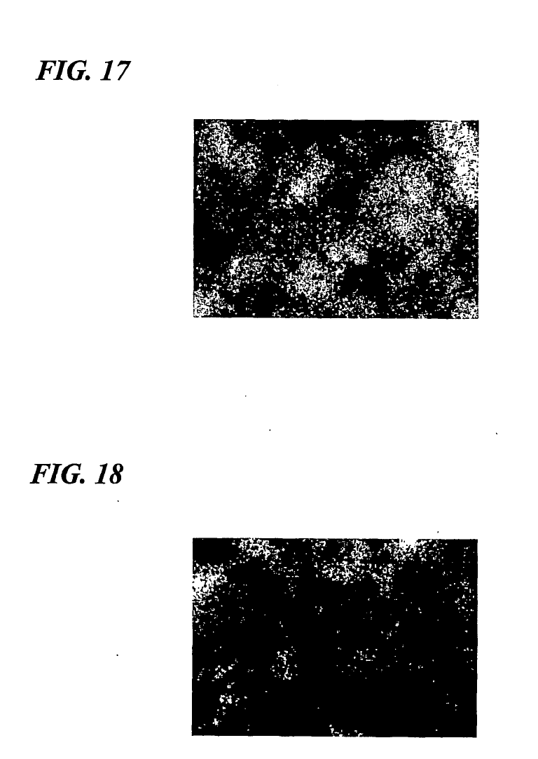

The chemical morphology NIH-based image contrast plots for the study, appearing in FIGS. 17-20, are as follows:

1. FIG. 17 is an agglomerated reference sample, microscopic NIR sucrose map, in which lighter areas indicate regions of sucrose. The grayscale magnitude of each pixel is directly related to the sucrose concentration and the region represented by each pixel. The field of view is 320×240 microns.

2. FIG. 18 is an agglomerated pilot sample, microscopic NIR sucrose map, in which lighter areas indicate regions of sucrose. The grayscale magnitude of each pixel is directly related to the sucrose concentration and the region represented by each pixel. The field of view is 320×240 microns.

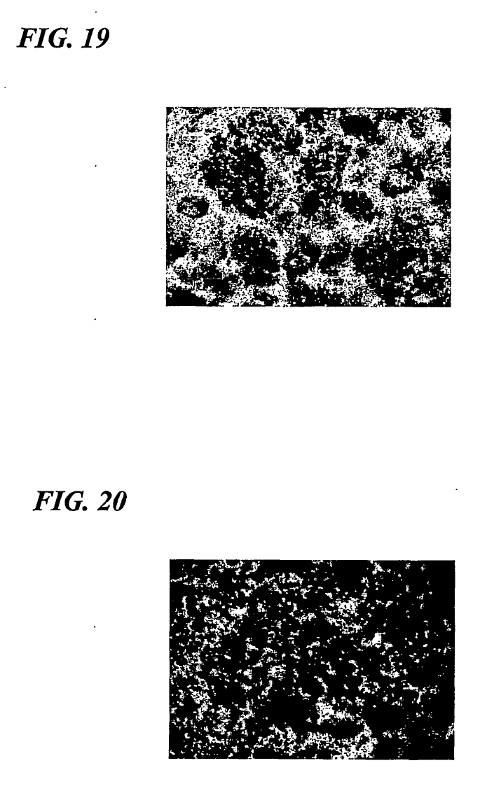

3. FIG. 19 is an agglomerated reference sample, macroscopic NIR sucrose map, in which lighter areas indicate higher areas of sucrose. The grayscale magnitude of each pixel is directly related to the sucrose concentration and the region represented by each pixel. The field of view is 3.375×2.475 mm. (This is termed a macroscopic NIR map; (a) because one dimension was greater than 3 mm; and (b) because by comparison to the scale of FIGS. 17 and 18 it is on a much larger scale and concerns general features. It actually fits close to the border between microscopic and macroscopic, as defined herein and is technically microscopic (<9 sq. mm) by those definitions.

4. FIG. 20 is an agglomerated pilot sample, macroscopic NIR sucrose map, in which lighter areas indicate higher areas of sucrose. The grayscale magnitude of each pixel is directly related to the sucrose concentration and the region represented by each pixel. The field of view is 3.375×2.475 mm.

In light of the differences seen in the average field of view spectra, hyperspectral image data were processed to yield the “sucrose maps.” FIGS. 17-20 show the microscopic and macroscopic hyperspectral image data as grayscale images, where the gray scale intensity of a given image pixel (lighter shade indicates greater magnitude) is directly proportional to the sucrose content in the pixel.

FIGS. 17 and 18 showing the microscopic field of view the sucrose maps exhibit a significant difference between the reference and the pilot agglomerated cocoas.

The macroscopic NIHI images (FIGS. 19 and 20) provide information more representative of the bulk properties of the cocoa powders as the domain size of each powder is considerably smaller than the field of view presented in each image. FIGS. 19 and 20 are the macroscopic sucrose map images, presented after identical data treatment as the microscopic images.

The macroscopic sucrose maps (FIGS. 19 and 20) indicate that the reference sample is of significantly higher sucrose concentration than the other agglomerated cocoas. The macroscopic images show greater sucrose signal in what could be termed the grain boundaries between individual particles. The reference sample (FIG. 19) shows significant “grain” structure, indicating a greater degree of exposure of the sucrose to the solution phase upon addition of the powder to liquid.

This analysis indicates, from the microsopic and macroscopic fields of view, that the primary difference between the agglomerated cocoa powder samples is the sucrose content. These data show that the agglomerated reference sample has a higher average sucrose content as well as a greater distribution of sucrose in both the microscopic and macroscopic domains. The differing amounts of sucrose, as well as the degree exposure of the sucrose to liquid, are factors that will affect the dispersion properties of the final cocoa powder. This difference may account for the initial differences in dispersability between cocoa powders. This data suggests that the fat removal process is not the primary contributor to differences in cocoa powder performance in milk and/or water.

To summarize, several cocoa powder samples were analyzed by Near Infrared Hyperspectral Imaging covering both microscopic and macroscopic domains. The goal of the study was to find differences in the samples that correlate with dispersability and wetting characteristics of the cocoa powders in milk or water. NIHI data show that all agglomerated cocoa powders contained sucrose and the reference agglomerated cocoa containers a higher concentration of sucrose than the pilot cocoa powder. Sucrose concentration and distribution is the only significant difference between the cocoa powders and is most likely the most significant factor affecting dispersion properties of the cocoas in aqueous systems.

### EXAMPLE 5

### Analysis of Beef Samples

Chemical imaging was applied to the analysis of beef (meat) samples with the aim of providing additional quantitative information regarding the quantity and distribution of fat in the muscle tissue. Near Infrared Hyperspectral Imaging in the macroscopic domain was applied to raw and cooked beef samples, where images obtained in the NIR region of the spectrum are compared with Bright field visible images. The regions of the beef that are primarily higher in fat content will show enhanced contrast in a chemical image than for the corresponding visible image. In this way, NIR hyperspectral imaging was used to unequivocally distinguish between the fat and lean tissue for a given sample.

Beef samples were prepared as raw and cooked and sectioned for analysis. The samples were illuminated with sufficient NIR radiation to produce spectral contrast in the wavelength regions of interest. The near infrared macroscopic imaging system produced images covering a filed of view of approximately 25 mm by 20 mm. Corresponding Bright field images were acquired with a stand mounted digital photographic camera and converted to gray scale images for display in this disclosure. (In the actual experiment colored images to evidence contrast were created.)

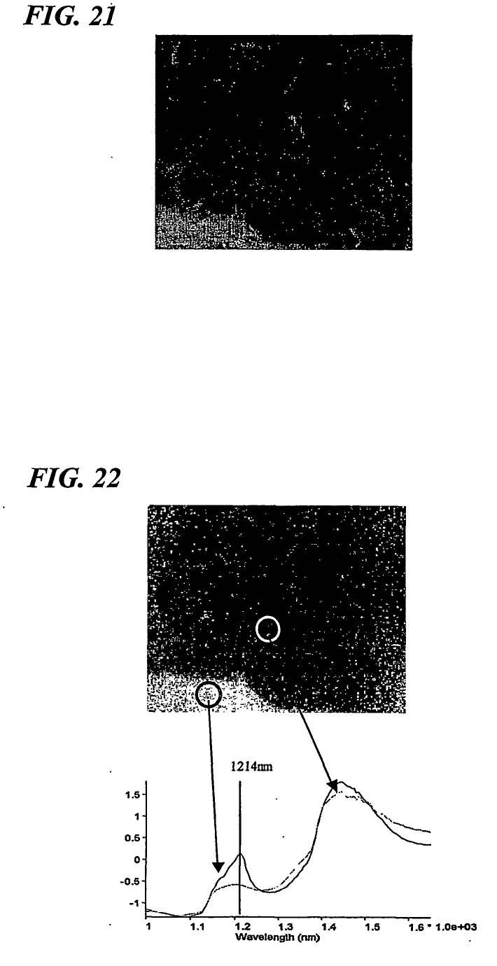

Because this technology is specific to the chemical compounds like fat and the proteins of the meat, regions containing different concentrations of such compounds may be distinguished. The data is truly unique because a vibrational spectrum unique for each chemical component in the meat is spatially provided for each pixel in the image field of view. FIGS. 21-24 illustrate the results of visible and NIR imaging for the analysis of meat samples. From these figures it is clear that regions of fat and marbling are visible with greater contrast in the NIR images relative to the visible images.

The figures from this study are as follows:

1. FIG. 21 is a visible grayscale image of raw rib eye steak (approximately 25 mm×20 mm field of view).

2. FIG. 22 is a grayscale image (chemical morphology NIR-based image contrast plot of raw rib eye steak taken from the 1214 nanometer image slice from the hyperspectral image of the beef. Regions of higher fat are clearly visible in this image (approximately 25 mm×20 mm field of view).

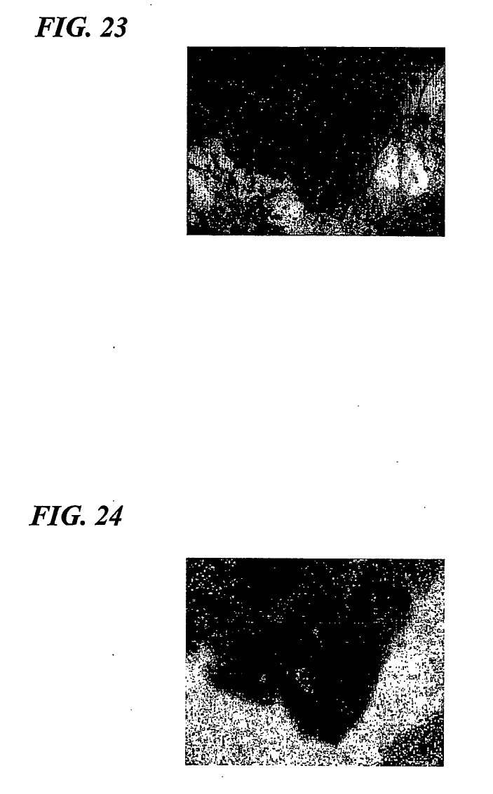

3. FIG. 23 is a visible grayscale image of cooked rib eye steak (approximately 25 mm×20 mm field of view).

4. FIG. 24 is a grayscale image (chemical morphology NIR-based image contrast plot) of cooked rib eye steak taken from the 1214 nanometer image slice from the hyperspectral image of the beef Regions of higher fat are clearly more visible in this image than in the Bright field image (approximately 25 mm×20 mm field of view).

### EXAMPLE 6

### Near Infrared Imaging of Barley Kernels

In sum, Near Infrared Hyperspectral Imaging (NIHI) was applied to whole kernel samples of seeds (barley) having high and low germination rates. Full spectral NIR image cubes were acquired of different kernel collections under identical illumination conditions. These image cubes were processed by spectral ratio methods (1200 nm image divided by the 1450 nm image) that resulted in increased contrast between oil rich and water rich regions. These resulting images were then analyzed for features that could be correlated with germination rate obtained from a post-image acquisition germination test. 85% of the non-germinating and 14% of the germinating kernels exhibited a sharp interface between the endosperm and the embryo in the ratio images. The presence of the endosperm/embryo interface in the absorbance ratio image is therefore correlated with a kernel's inability to germinate. This type of image analysis may be used to predict germination quality in a batch prior to processing.

Near infrared imaging technology was applied to barley samples classified based on the percentage of kernels that germinate after a defined time period. Laboratory analysis was performed on barley lots in order to provide wet chemical analysis data that may be compared with spectroscopic results. The goal of this work is the determination of any correlation between hyperspectral imaging results and the germination potential of individual barley grains from lots classed as “good” and “bad” from a germination perspective.

Thirty barley samples representing seven different varieties were obtained. The samples were analyzed by laboratory methods and a spreadsheet detailing the results was obtained. The following parameters are significant in determining the quality o the barley for malt processing: moisture and protein content, as measured by NIR spectroscopy, α-amylase activity and percent germination before and after aging. Two samples were selected from the same variety (Metcalfe) for NIR imaging: samples RE-0032 and RE-0033, representing high and low germination rates, respectively. Table 2 details the laboratory results for these two samples.

TABLE 5
Laboratory Analysis Results For Two Barley Samples Used in NIR
Imaging Analysis

| | | | % | | | % Germ | Chitted |
|---|---|---|---|---|---|---|---|
| | | % | Protein, | α-amylase | | After | %, by |
| | | Moisture, | db by | Activity/g | % Germ | Aging | Perling |
| Sample # | Variety | by NIR | NIR | Dry IDC/g | Jan. 18, 2001 | Feb. 9, 2002 | Test |
| RE 00- | Metcalfe | 12.8 | 10.4 | 60 | 99 | 99 | 0 |
| 32 | | | | | | | |
| RE 00- | Metcalfe | 15.0 | 11.4 | 2389 | 89 | 22 | 13 |
| 33 | | | | | | | |

Sample RE-0033 (sample 33) is classified as a “bad” barley sample due to the low germination rate, and this is clearly anti-correlated with alpha amylase activity. In addition, sample 33 has higher moisture content as well as a slightly higher protein content relative to sample 32. NIR hyperspectral images (chemical morphology NIR-based image contrast plots) were obtained for a collection of kernels representing each sample, and these kernels were subsequently germination tested in order to generate a correlation between spatial and spectral features and germination potential.

NIR Hyperspectral Macroscopic Imaging

Barley kernels were taken from each 50-gram bag of barley kernels in a random fashion and arranged on a Spectralon™ low reflectivity NIR reference standard. Initial experiments were performed on randomly arranged, un-numbered kernels. NIR imaging experiments using kernels that would be germination tested were performed with 17 kernels from each sample batch oriented either ridge up or ridge down with the embryo ends aligned. The kernels were numbered from 1 to 17 and placed into individual vials between imaging runs in order to retain the kernel registration. Digital RGB images were captured of each collection of kernels and used for comparison and identification.

The NIR system was equipped with standard camera optics and therefore some image aberration is evident as the system is scanned from 1075 nm to 1650 nm. However, the focus and zoom were set using a probe wavelength of 1380 nm in order to assure best focus throughout the scan. Images were collected as 100 scan averages at each wavelength under 90-watt tungsten halogen ring light illumination. Reference images of a high reflectivity Spectralon™ reference material were acquired after each sample scan and saved. Each image was dark corrected (single archived dark scan) and transformed, using the corresponding reference image, into an absorbance image and inspected for contrast and features in both the spatial and spectral domains.

Seed Germination

After collection of NIR and RGB images, the individual grains from each batch were germinated in glass vials at room temperature for 72 hours. To each vial was added a single kernel placed between two moist cotton balls. The kernels were removed for inspection at 24, 48, and 72 hours and the germination state recorded. In this way, individual kernel germination results can be mapped to the hyperspectral image for each kernel.

Initial Imaging Results

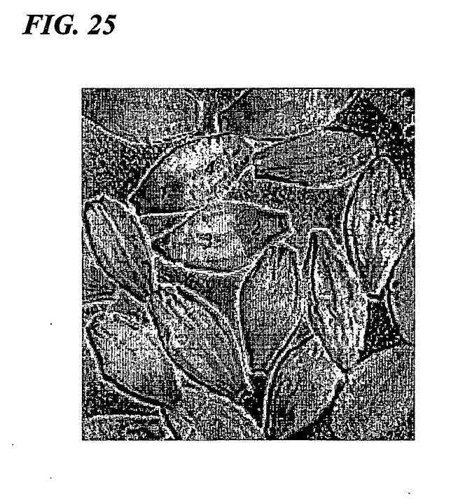

Initial attempts to obtain hyperspectral images of non-oriented grains generated data that were used to further refine the data collection parameters. Initial results indicated that some structural differences were evident between good and bad barley, but these could not be correlated with individual grains as no germination test was performed. Specifically, a small number of grains showed a separation or contrast between the embryo and endosperm at several wavelengths in the normalized (spectral domain) images. The normalized 1280 nm image (i.e., chemical morphology NIR-based image contrast plot) is shown in FIG. 25. (FIG. 25 is an NIR normalized image (1380 nm) of barley grain from sample RE 00-32 showing spectral contrasts between embryo and endosperm, the numbers 1 and 2 designate the germ and numbers 3- and 4 designate the endosperm, in two selected kernels.) It appears from these data, that the embryo regions exhibit lower oil content relative to the endosperm. As the kernels were arranged randomly and not germination tested, these data could not be correlated with germination rate. However, the data suggests a difference between kernels that may be related to germination rate.

Several kernels in the center of the image display a significant contrast between the endosperm and the embryo in the ridge down orientation. The contrast was not evident in Bright field visible images of the kernels and this indicates that NIR diffuse reflectance absorption path length was sufficient to penetrate the husk and allow diffuse imaging of the material directly underneath. The endosperm/embryo spectral contrast is greatest at 1380 nm, which is the first overtone of the CH₂ combination band absorption region. However, other regions of contrast are the second overtone CH stretch region around 1200 nm as well as the second overtone OH stretch between 1400 and 1450 nm. In addition, the regions specified here are also anti-correlated with respect to functional group: higher OH signal is in regions of lower CH (or CH₂) functionality. This indicates a separation of OH and CH primary functional group constituents across the endosperm interface.

Germination Test and NIR Imaging Results

The preliminary imaging data led to a conclusion that a germination test was needed in order to find a correlation between spectral and spatial features and germination potential. To this end, only two samples (high and low germ rate) were used in the final data collection experiment, where 17 kernels from each sample were imaged under identical illumination and orientation conditions. The orientation and kernel numbering for each batch is shown in the RGB (converted to grayscale images for this disclosure; numbering is right to left for each row, bottom to top for sequential rows) images presented in FIGS. 26-29.

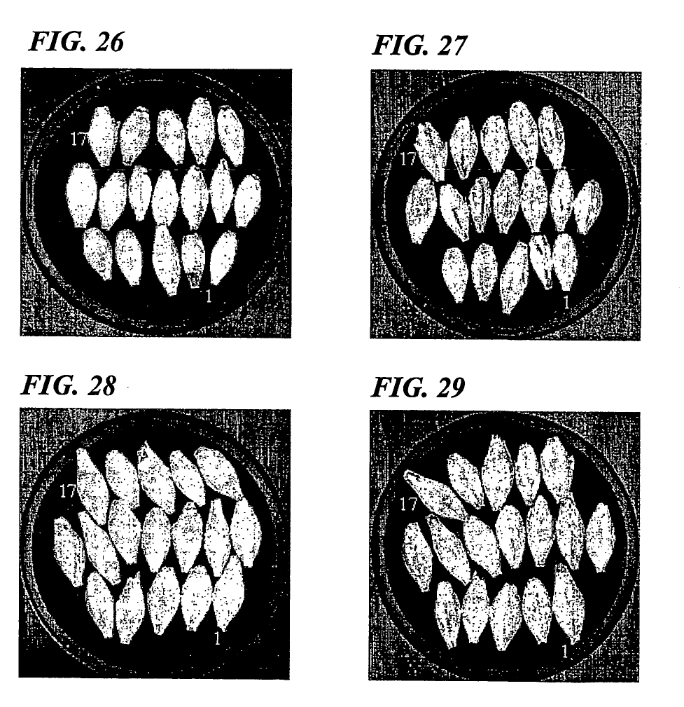

In FIG. 26, sample RE 00-32 is shown ridge side down. In FIG. 27, sample RE 00-32 is shown ridge side up. In FIG. 28, sample RE 00-33 is shown ridge side down. In FIG. 29, sample RE 00-33 is shown ridge side up. Numbers are placed in the RGB images (FIGS. 26-29) to identify specific barley kernels.

Kernel sizing was performed on the calibrated RGB image using Image Pro Plus™. The kernels from Sample RE 00-32 have an average length of 0.30 inches (α=0.03 in.) and width 0.14 (α=0.01 in.). The more uniform sample RE 00-33 kernels exhibited slightly larger average kernel length of 0.35 inches (α=0.02 in.), and width of 0.15 inches (α=0.009 in.). It is unclear if the slight average size difference between samples is a factor in germination quality.

The germination rate for sample RE 00-32 is 76.5%, where kernels 2, 14, 15, and 16 did not germinate after 72 hours. Kernel 2 is clearly visibly darker in the embryo region indicating damage to the kernel. Of the seeds from sample RE 00-32 that germinated, kernels 1, 3, 6, 10, 12, and 13 required greater than 24 hours to germinate. Only kernel 2 in sample RE 00-33 germinated. These results agree with the assessments presented in the laboratory data, although the germination rates are slightly lower in the test data than in the laboratory data. This may be due to the small sample set in our test relative to the laboratory germination tests (17 vs. 100 kernels) or to uncontrolled variables in our germination test (from temperatures subject to HVAC cycling). Full germination data are included in Tables 6 and 7.

After collection of the RGB image for each sample, an NIR image from 1075 to 1625 nm was collected. After collection, the image cubes were dark corrected and converted to absorbance images. Several image-processing methods were applied to the data, including normalization and derivative processing in the spectral domain. However, a simple ration between the CH absorption peak (1200 nm) and the OH absorption peak (1450) yields images that have significant contrast between the endosperm and embryo region s for a significant number of kernels in sample RE 00-33.

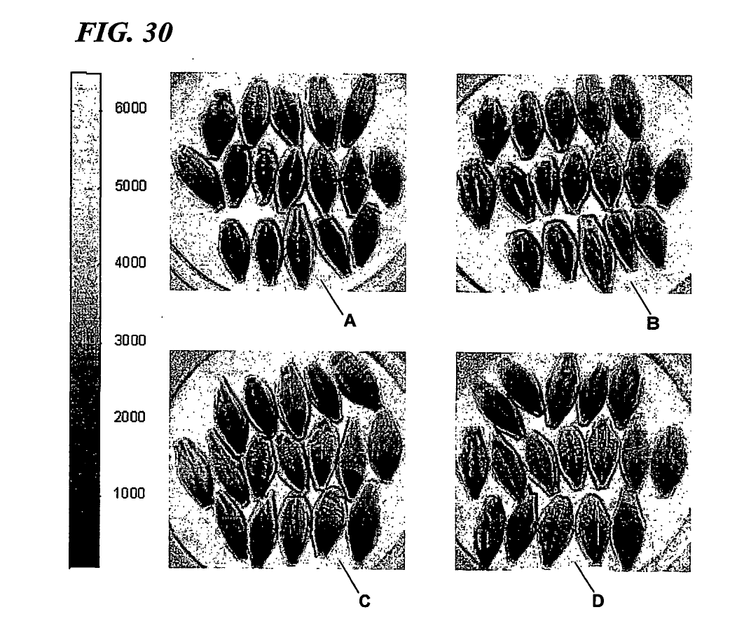

The images in FIG. 30 show a significant difference between the kernels classed as “good” and “bad.” In FIG. 30, barley sample image is derived from NIR absorption ridge cubes (i.e., chemical morphology NIR-based image contrast plots) are shown. In FIG. 30, four images are shown. The image indicated at A is of sample RE 00-32 ridge side down. The image of B is of sample RE 00-32 ridge side up. The sample in C is RE 00-33 ridge side down. The image at D is RE 00-33 ridge side up. Each image is the ratio of the 1200 nm absorbance image and the 1450 nm absorbance image converted to 16 grayscale for visualization. The grayscale is depicted in the left side of the picture. The ratio images are representative of the distribution of oil and water rich regions in the kernels. Low grayscale values are regions of higher OH content, with a ratio of A₁₂₀₀ to A₁₄₅₀ is low. The contrast between the endosperm and embryo is not clearly seen in the RE 00-33 sample of kernel images (ridge side down), where a majority have a significant delineation between the regions.

In general, greater differences between images are visible in the ridge down images, where greater embryo visibility underneath the husk is afforded. Therefore, the following analysis will compare the ridge down images. Sample RE 00-33, the low percentage germination sample, exhibits significant embryo interface contrast in almost every kernel in the ridge down ratio image. Only kernel 2 germinated, and this occurred within the first 24 hours. Two kernels, 4 and 12, do not have as sharp an interface as the rest of the kernels, whereas kernel 2, which did germinate, displays the characteristic sharp contrast across the embryo interface. These differences are indicative of the presence of more than one process or factor that affects germination. More importantly, of the 16 kernels that did not germinate, 14 exhibited qualitatively sharp contrasts between a visible separation of the endosperm and embryo in the ratio image and the ability of the kernel to germinate.

The higher percentage germination sample, #32, possesses three kernels that display a significant contrast at the embryo interface in the ridge down ratio image. Of these, two did not germinate (14 and 16). Kernels 2 and 15 do not show these features although they did not germinate. The only other kernel that displays significant contrast at the embryo interface is kernel 13, which germinated between 24 and 48 hours. The kernels that did not germinate or had delayed germination exhibit, in general, a sharper contrast between the endosperm and embryo regions. Kernels 2 and 15, however, do not fit this trend, and this is again indicative of more than one factor affecting the germination success in a batch of kernels.

Of the 34 kernels in the experiment, 20 did not germinate and 85% of these kernels exhibited the sharp interface between the embryo and endosperm. Likewise, 14% of the germinating kernels exhibited the sharp interface. The assessment of the “sharpness” of the interface region is qualitative at best and influenced by the image depth of field and illumination parameters. However, a qualitative judgment regarding the presence of a “sharp” interface can be used to develop a correlation between germination quality and spatial features in the ratio images.

The presence of a visible interface between the embryo and endosperm in the ratio images indicates a separation of CH and OH bearing constituents in the kernel prior to full germination. The germination process converts, by enzymatic activity, starch located in the endosperm into maltose that is used as an energy source for the growing embryo. Germination is initiated by absorption of water through the husk (imbibition) that in turn activates the diastatic enzymes. The lower CH to OH band ratio in regions corresponding to the embryo region indicates enhanced hydration in the embryo. This indicates the germination process was begun and then arrested prior to receipt. The alpha amylase activity in the dry RE 00-33 barley is several orders of magnitude greater than that of the RE 00-32 sample. This also indicates that the enzymatic activity was initiated but did not result in full germination due to drying of the endosperm or some other environmental effect.

In conclusion, two barley samples, RE 00-32 and RE 00-33, were analyzed by NIR hyperspectral imaging for features and structures that may correlate with germination rate for each batch. The samples were of the Metcalfe variety and were imaged ridge up and ridge down using the NIR and RGB (visible) imaging systems. Image cubes for each sample were processed to yield grayscale images where the gray level at each pixel is representative of the ratio of the CH and OH optical absorption bands. 85% of non-germinating kernels and 14% of the germinating kernels in the experiment exhibited a sharp interface between the endosperm and the embryo in the ridge up ratio images. Therefore, a strong correlation exists between the germination rate and the presence of a defined interface between the endosperm and embryo regions. NIR hyperspecteral imaging has demonstrated utility in the evaluation of the germination quality of barley prior to germination and may be useful for application in grain quality assessment after additional research effort.

TABLE 6
Barley Sample RE 00-32 72-hour germination test results

| Kernel Number | 24 Hours | 48 Hours | 72 Hours |
|---|---|---|---|
| 1 | No | Yes | Yes |
| 2 | No | No | No |
| 3 | No | No | Yes |
| 4 | Yes | Yes | Yes |
| 5 | Yes | Yes | Yes |
| 6 | ? | ? | Yes |
| 7 | Yes | Yes | Yes |
| 8 | Yes | Yes | Yes |
| 9 | Yes | Yes | Yes |
| 10 | No | Yes | Yes |
| 11 | Yes | Yes | Yes |
| 12 | ? | Yes | Yes |
| 13 | No | Yes | Yes |
| 14 | No | No | No |
| 15 | No | No | No |
| 16 | No | No | No |
| 17 | Yes | Yes | Yes |

TABLE 7
Barley Sample RE 00-33 72-hour germination test results

| Kernel Number | 24 Hours | 48 Hours | 72 Hours |
|---|---|---|---|
| 1 | No | No | No |
| 2 | Yes | Yes | Yes |
| 3 | No | No | No |
| 4 | No | No | No |
| 5 | No | No | No |
| 6 | No | No | No |
| 7 | No | No | No |
| 8 | No | No | No |
| 9 | No | No | No |
| 10 | No | No | No |
| 11 | No | No | No |
| 12 | No | No | No |
| 13 | No | No | No |
| 14 | No | No | No |
| 15 | No | No | No |
| 16 | No | No | No |
| 17 | No | No | No |

What is claimed is:

1. A method of evaluating a selected agricultural or food material; said method comprising a step of:
(a) preparing a chemical morphology NIR-based image contrast plot of a region of the selected material having an area of no greater than 50 cm by 50 cm.

2. A method according to claim 1 wherein:
(a) said step of preparing a chemical morphology NIR-based image contrast plot is conducted to plot distribution of an additive in a material.

3. A method according to claim 2 wherein:
(a) said step of preparing a chemical morphology NIR-based image contrast plot is conducted to plot distribution of an additive in a food material.

4. A method according to claim 1 wherein:
(a) said step of preparing a chemical morphology NIR-based image contrast plot is conducted to plot distribution of an additive in paper.

5. A method according to claim 1 wherein:
(a) said step of preparing a chemical morphology NIR-based image contrast plot is conducted to plot distribution of a modified seed based fiber in a material.

6. A method according to claim 1 wherein:
(a) said step of preparing a chemical morphology NIR-based image contrast plot is conducted to plot distribution of fat in a material.

7. A method according to claim 1 wherein:
(a) said step of preparing a chemical morphology NIR-based image contrast plot is conducted to plot distribution of effect of an additive in a plant material.

8. A method according to claim 7 wherein:
(a) said step of preparing a chemical morphology NIR-based image contrast plot is conducted to evaluate distribution of effects of an additive selected from: herbicide(s); pesticide(s); and fertilizer(s), or mixtures thereof, in plant material.

9. A method according to claim 1 wherein:
(a) said material used, in said step of preparing a chemical morphology NIR-based image contrast plot of a selected material, is plant material.

10. A method according a claim 1 wherein:
(a) said material used, in said step of preparing a chemical morphology NIR-based image contrast plot of a selected material, is animal material.

11. A method according to claim 1 wherein:
(a) said steps of preparing a chemical morphology NIR-based image contrast plot is conducted to plot distribution of fat in a cocoa powder.

12. A method according to claim 1 wherein:
(a) said step of preparing a chemical morphology NIR-based image contrast plot is conducted to plot distribution of fat above a selected % content in a material.

13. A method according to claim 1 wherein:
(a) said step of preparing a chemical morphology NIR-based image contrast plot is conducted to plot distribution of water in a material.

14. A method according to claim 13 wherein:
(a) said step of preparing a chemical morphology NIR-based image contrast plot is conducted to plot distribution of water in an emulsion.

15. A method according to claim 14 wherein:
(a) said step of preparing a chemical morphology NIR-based image contrast plot is conducted to plot distribution of water in a chicken skin emulsion.

16. A method according to claim 1 wherein:
(a) said step of preparing a chemical morphology NIR-based image contrast plot of the selected material comprise preparing a contrast plot representing a sample area of no greater than 3 mm×3 mm.

17. A method according to claim 1 wherein:
(a) said step of preparing a chemical morphology NIR-based image contrast plot is conducted to show distribution of a component in a material wherein the component comprises no more than 5%, by wt., of the material.

18. A method according to claim 17 wherein:
(a) said step of preparing a chemical morphology NIR-based image contrast plot is conducted to show distribution of a component in a material wherein the component comprises no more than 2%, by wt., of the material.

19. A method according to claim 1 wherein:
(a) said step of preparing a chemical morphology NIR-based image contrast plot comprises a step of collecting NIR data by diffuse reflectance spectroscopy.

20. A method according to claim 1 wherein:
(a) said step of preparing a chemical morphology NIR-based image contrast plot comprises a step of plotting a result of NIR data collected from an overtone and over a wavelength range of 200 nm or less.

21. A method according to claim 1 wherein:
(a) said step of preparing a chemical morphology NIR-based image contrast plot includes a step of collecting NIR data from a sample of the material having a thickness of no more than 100 microns.

22. A method according to claim 1 wherein:
(a) said step of preparing a chemical morphology NIR-based image contrast plot is conducted to plot distribution of a sugar in a material.

23. A method according to claim 22 wherein:
(a) said step of preparing a chemical morphology NIR-based image contrast plot is conducted to plot distribution of sucrose in a material.

24. A method according to claim 23 wherein:
(a) said step of preparing a chemical morphology NIR-based image contrast plot is conducted to plot distribution of sucrose in cocoa powder.

25. A method according to claim 1 wherein:
(a) said step of preparing a chemical morphology NIR-based image contrast plot is conducted to show distribution of fat in meat.

26. A method according to claim 25 wherein:
(a) said step of preparing a chemical morphology NIR-based image contrast plot is conducted to show distribution of fat in beef.

27. A method according to claim 1 wherein:
(a) said step of preparing a chemical morphology NIR-based image contrast plot is conducted to evaluate oil and water rich regions in a seed.

28. A method according to claim 27 wherein:
(a) said step of preparing a chemical morphology NIR-based image contrast plot is conducted to evaluate oil and water rich regions in a barley kernel.

29. A chemical morphology NIR-based image contrast plot made according to the method of any one of claims 1-28.
